SEQUENCE LISTING (7 sequences)

SEQ ID NO: 1 - DNA, 1970 bp, Rattus norvegicus
acatgggcacgcgtggtcgacggcccgggctggctgggcaacacgggttcagcccaggtttcatagtaagttccagacactcctggaaaaacaatacaggtccctgacaaaagaaaaaacaaaacaaaggaaacagaaacatgcgtttttaaaaaagaaggaggagactccatgaaggcaggccttgggtggggtcactgcttctctgtacacaggaggagaattgccaagatcttccggacagtgtggactatactgtaagaccctctcaatacagacagactggacaggcatagtgacacatgcctttaatgcctgcagtactcaggaggaggtggcaggtggaacggctgttctttgaggttcaagaccagcgtggactacagagtgagttccaggacaggcagggctacacagaaaaatcctgtctgaaaacaaaacaaaacccagacagacacaccaaaaacagccaagggaccagagagatgggtcagggcctaatcacttgctactctttgcagaggacccaaatttagttcctataaccctccatgagaagcttcacaattgtctctaactcaattccacccgtgttccgacctcccatatgcaccagacatgttatactcacacatacgcacaaacacacacacacacacacacacacacacacacacacacacacacacacggaaaacatataaaataaagatttaaaaaatctttttcttttggccggggtgtgtgggagagcatctgagccatctcaccagcccagggtgcacgtctttttctttttttcggagctggggaccgaacccagagccttgtgcttgctaggcaagtgctctaccactgagctaaatccccaaccccggagcacgtctttaatcccagaatcaggaggtagaggtaatgagatcccagtgagcccaaggtcagccgagtctacaaagtgagttccaggacagccagaactaatcttggaaaaacaaacaagggctggtgaggtggttcagtagttaagaacactggctgctcttccagaggtcctgagttcattctcagtaaccacatggtggggatctgatgcctgttctggcatgcagatatacatgcagatagtgcactcctacatttaaaaaaaaaagacataaataatattttaaaacattgggcgttttgtcttctaataaaacttcactgctatcttctaataaaaattcactgctagccgcggggtgtggtgcccccatacctttaatcccaacaacttgagaggcagaggcaggcggacctttgagtttgaagctagcctggtctacagagtgagttcaagatagccacggatagtcagaaagtcctgtttcgaacctctccccaaccaaatcactcctgtaatcccagcactctggaggcagtagcaggttagtccctgcttctcagagagaggagagagagagagagagagaggagacacacacacacagagacagagaggagagagaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 2 - DNA, 464 bp, Artificial Sequence
  synthetic oligonucleotide
gaaagagaaagagaatgggacagcatgtgactgcctgatgaagttggcgtgcttgctcaaaagttctgcgagattgacggctctctggatttgagccaaggacacgcctgggaagccacggtgacctcacaaggcccggaatctccgcgagaatttcagtgttgttttcctctctccacctttctcagggacttccgaaactccgcctctccggtgacgtcagcatagcgctgcgtcagactataaactcccgggtgatcgtgttggcgcagattgactcagttcgcagcttgtggaagattacatgcgagaccccgcgcgactccgcatccctttgccgggacagcctttgcgacagcccgtgagacatcacgtccccgagccccacgcctgagggcgacatgaacgcgctggccttgagagcaatccggacccacgatcgcttttggcaaaccgaaccggac

SEQ ID NO: 3 - DNA, 23 bp, Artificial Sequence
  synthetic oligonucleotide primer
ccctctgctaaccatgttcatgc

SEQ ID NO: 4 - DNA, 23 bp, Artificial Sequence
  Synthetic oligonucleotide primer
tcttgctcacgaatacgacggtg

SEQ ID NO: 5 - DNA, 21 bp, Artificial Sequence
  Synthetic oligonucleotide primer
ctggtctacagtaacgggtcc

SEQ ID NO: 6 - DNA, 19 bp, Artificial Sequence
  Synthetic oligonucleotide primer
attcagcccatatcgtttc

SEQ ID NO: 7 - DNA, 508 bp, Artificial Sequence
  Synthetic sequence of AEG-1 promoter
ctggtctacagtaacgggtccccaacgccgcgtcttaaccaggcccgaaccgaccaccgccaaccttccctgacacgcctttgccccacccgaccccgcccgcccccacgtgacgcccacgccccgcccactggcgcccacgtgacccacggtcgtccccgcgcgcggcgtggatcgcggcccaagccgccattgttccgccgagggaggacagcggggcctggcgctggcgccgagacgccgcttagcggccgccactggagacactccctcccgcctcccgggtctcctggcggcggcggagtgaggctgacagcggggaacctgggagacccctccgccctccccgcggtggcagcggccgatccccggctccggcgcgagggacggccgcgatgcgctcggcctgaggttacccggcccggcccttcctcgcttccctcgactattccactgcgtctccgcgccccggcgtcatcctgcgagtccctctgacgggagggaagat

(19) United States
(12) Patent Application Publication
(10) Pub. No.: US 2014/0227182 A1
(43) Pub. Date: Aug. 14, 2014

(54) CANCER IMAGING WITH THERAPY: THERANOSTICS

(71) Applicants: Martin Gilbert POMPER, Baltimore, MD (US); Hyo-eun BHANG, Baltimore, MD (US); Paul FISHER, Richmond, VA (US)

(72) Inventors: Martin Gilbert POMPER, Baltimore, MD (US); Hyo-eun BHANG, Baltimore, MD (US); Paul FISHER, Richmond, VA (US)

(73) Assignees: THE JOHNS HOPKINS UNIVERSITY, Baltimore, MD (US); VIRGINIA COMMONWEALTH UNIVERSITY, Richmond, VA (US)

(21) Appl. No.: 14/182,690

(22) Filed: Feb. 18, 2014

Related U.S. Application Data
(63) Continuation-in-part of application No. 13/881,777, filed on Jun. 13, 2013.
(60) Provisional application No. 61/766,473, filed on Feb. 19, 2013.

(51) Int. Cl. A61K 51/04; A61K 48/00; A61K 49/00

(52) U.S. Cl. CPC A61K 51/0491; A61K 49/0054; A61K 48/0058
USPC 424/1.73; 424/9.6; 514/44 R

(57) ABSTRACT

Genetic constructs comprising reporter genes operably linked to cancer specific or cancer selective promoters (such as the progression elevated gene-3 (PEG-3) promoter and astrocyte elevated gene 1 (AEG-1) promoter) are provided, as are methods for their use in cancer imaging, cancer treatment, and combined imaging and treatment protocols, e.g. for imaging and/or treating spontaneous metastasis. Transgenic animals in which a reporter gene is linked to a cancer specific or cancer selective promoter, and which may be further genetically engineered, bred or selected to have a predisposition to develop cancer, are also provided.

### BACKGROUND OF THE INVENTION

1. Field of the Invention

The invention generally relates to genetic constructs and methods for their use in cancer imaging, cancer treatment, and combined imaging and treatment protocols, e.g. for imaging and/or treating spontaneous metastasis. In particular, transcription of genes in the constructs is driven by cancer specific or cancer selective promoters.

2. Background of the Invention

Targeted imaging of cancer remains an important but elusive goal. Such imaging could provide early diagnosis, detection of metastasis, aid treatment planning and benefit therapeutic monitoring. By leveraging the expanding list of specific molecular characteristics of tumors and their microenvironment, molecular imaging also has the potential to generate tumor-specific reagents. But many efforts at tumor-specific imaging are fraught by nonspecific localization of the putative targeted agents, eliciting unacceptably high background noise.

While investigators use many strategies to provide tumor-specific imaging agents—largely in the service of maintaining high target-to-background ratios—they fall into two general categories, namely direct and indirect methods¹. Direct methods employ an agent that reports directly on a specific parameter, such as a receptor, transporter or enzyme concentration, usually by binding directly to the target protein. Indirect methods use a reporter transgene strategy, in analogy to the use of green fluorescent protein (GFP) in vitro, to provide a read-out on cellular processes occurring in vivo by use of an external imaging device. Molecular-genetic imaging employs an indirect technique that has enabled the visualization and quantification of the activity of a variety of gene promoters, transcription factors and key enzymes involved in disease processes and therapeutics in vivo including Gli², E2F1³, telomerase^{4,5}, and several kinases, including one that has proved useful in human gene therapy trials^{6,7}. Unfortunately, to date, none of these techniques has provided sufficient specific localization of imaging agents, and unacceptably high background noise is still prevalent.

Cancer therapies have also advanced considerably during the last few decades. However, they are also still hampered by nonspecific delivery of anti-tumor agents to normal cells, resulting in horrendous side effects for patients. This lack of specificity also results in lower efficacy of treatments due to the want of a capability to deliver active agents in a focused manner where they are most needed, i.e. to cancer cells alone.

In addition, there is currently no efficacious way to image and/or treat cancerous cells and tissues which are caused by spontaneous metastasis. Spontaneous metastasis refers to cancer cells that escape from the primary tumor, enter the bloodstream or lymphatics, that settle in tissue remote from the primary tumor and grow and thrive in the new location(s). For example, prostate cancer often metastasizes to bone.

U.S. Pat. No. 6,737,523 (Fisher et al.), the complete contents of which is hereby incorporated by reference, describes a progression elevated gene-3 (PEG-3) promoter, which is specific for directing gene expression in cancer cells. The patent describes the use of the promoter to express genes of interest in cancer cells in a specific manner. However, imaging and combined imaging and treatment are not discussed.

United States patent application 2009/0311664 describes cancer cell detection and imaging using viral vectors that are conditionally competent for expression of a reporter gene only in cancer cells. However, the technique is not used in vivo, combined methods of imaging and treatment are not discussed, and only herpes and vaccinia viruses are discussed in detail.

There is an ongoing need to develop improved methods of cancer imaging and treatment that are highly specific for cancer cells, and it would be a boon for patients and physicians to have available methods which combine a means of cancer imaging and a means of therapeutically treating cancer in a single method.

### SUMMARY OF THE INVENTION

The invention generally relates to genetic constructs and methods for their use in i) cancer imaging, and ii) cancer treatment; and iii) combined treatment and imaging. Combined treatment and imaging may be referred to herein as a “theranostic” approach to cancer. The gene constructs used in these methods comprise a promoter that is specifically or selectively active in cancer cells. These promoters may be referred to herein as “cancer promoters” or “cancer specific/selective promoters” or simply as “specific/selective promoters”. Due to the specificity afforded by these promoters, compositions, which include the constructs of the invention, can be advantageously administered systemically to a subject that is in need of cancer imaging or cancer treatment, or both. The promoters, e.g., AEG-Prom, are advantageously highly effective for imaging and treating spontaneous metastases, as demonstrated in Example 3 below.

The treatment aspect of the invention provides a high level of precise delivery of anti-tumor agents to cancer cells, even when delivery is made systemically, since the anti-tumor agents associated with the methods are only expressed within cancer cells. This advantageously results in few or no side effects for patients being treated by the method.

Similarly, the imaging aspect of the invention provides a high level of precise imaging of cancer cells and tumors with little or no background signal. Importantly, since there is little or no background “noise”, the imaging techniques of the invention enable the facile detection of metastatic cancer, even metastatic cancer that is not detectable with other methods due to e.g., the very small size of a newly developing tumor, or the diffuse pattern of cancer cells which do not actually form a tumor. As is well known in the art, early detection of tumors can significantly improve the outcome of tumor treatment. Similarly, detection of cancerous tissues before formation of a tumor will provide significant benefits.

The combined imaging and treatment methods are advantageous over the prior art in many ways. A combined approach to imaging and therapy is more efficient and requires fewer procedures, and hence less effort, on the part of the patient and the cancer specialist. Since activity is confined to cancer cells, side effects are reduced. In addition, the combined imaging and treatment method provides the ability to accurately monitor the effects of prior treatment concomitantly with providing treatment and this provides a cancer treatment specialist with an invaluable and accurate window on the progress of therapy, permitting therapeutic parameters to be fine-tuned in close conjunction with treatment.

In addition, the invention provides transgenic animals that have been genetically engineered to contain nucleotide sequences encoding a reporter gene operably linked to a cancer specific or cancer selective promoter, and their use for clinical evaluation of therapies. In some embodiments, the transgenic animals have a propensity for developing cancer.

It is an object of this invention to provide a method of imaging tumors or cancerous cells or tissue in a subject. The method comprises the steps of 1) administering to said subject a nucleic acid construct comprising an imaging reporter gene operably linked to a cancer specific or cancer selective promoter, 2) administering to said subject an imaging agent that is complementary to said imaging reporter gene; and 3) imaging tumors or cancerous tissues or cells in said subject by detecting a detectable signal from said imaging agent. In some embodiments, the imaging reporter gene is selected from the groups consisting of luciferase and herpes simplex virus 1 thymidine kinase (HSV1-tk); the subject may be a cancer patient. The imaging agent may be a radiolabeled nucleoside analog such as 2′-fluoro-2′deoxy-β-D-5-[¹²⁵I]iodouracil-arabinofuranoside. The step of imaging may be carried out via single photon emission computed tomography (SPECT) or by positron emission tomography (PET) The imaging reporter gene may be luciferase and said subject is a laboratory animal, in which case the imaging agent is a luciferase substrate. In some embodiments, the nucleic acid construct is present in a polyplex with a cationic polymer such as polyethylemeinine. One or both of the steps of administering may be carried out systemically. The step of administering a nucleic acid construct may be carried out by intravenous injection. In some embodiments, the tumors, cancerous tissues or cells include cancer cells of a type selected from groups consisting of breast cancer, melanoma, carcinoma of unknown primary (CUP), neuroblastoma, malignant glioma, cervical, colon, hepatocarcinoma, ovarian, lung, pancreatic, and prostate cancer. In some embodiments, the nucleic acid construct is present in a plasmid. In other embodiments, the nucleic acid construct is present in a viral vector such as a conditionally replication-competent adenovirus. In some embodiments, the cancer specific or cancer selective promoter is the progression elevated gene-3 (PEG-3) promoter.

The invention also provides a method of both imaging and treating tumors, or cancerous tissues or cells in a subject. The method includes the steps of 1) administering to said subject one or more nucleic acid constructs comprising an imaging reporter gene operably linked to a cancer specific or cancer selective promoter and a gene encoding an anti-tumor agent; 2) administering to said subject an imaging agent that is complementary to said imaging reporter gene; and 3) imaging tumors or cancerous tissues or cells in said subject by detecting a detectable signal from said imaging agent, wherein said gene encoding said anti-tumor agent is expressed by cells in said tumors or cancerous tissues or cells to act on said cells. In some embodiments, at least one, and possibly both, of the steps of administering may be carried out systemically. In some embodiments, the gene encoding an anti-tumor agent is operably linked to a tandem gene expression element, for example, an internal ribosomal entry site (IRES). In other embodiments, the gene encoding an anti-tumor agent is operably linked to a cancer specific or cancer selective promoter. The anti-tumor agent may be mda-7/IL-24 or tumor necrosis factor (TNF)-related apoptosis-inducing ligand (TRAIL).

The invention also provides a cancer specific or cancer selective gene expression imaging system, comprising a nucleic acid construct comprising an imaging reporter gene operably linked to a cancer specific or cancer selective promoter. In some embodiments, the cancer specific or cancer selective promoter is PEG-Prom. In some embodiments, the system is suitable for systemic administration.

The invention further provides a transgenic animal genetically engineered to contain and express a reporter gene linked to a cancer specific or cancer selective promoter. In some embodiments, the transgenic animal is also predisposed to develop cancer.

### BRIEF DESCRIPTION OF THE DRAWINGS

FIGS. 1A and B. PEG-Prom mediated reporter expression systems. A) Construct map of pPEG-Luc containing the firefly luciferase (Luc) encoding gene under the control of PEG-Prom; B) Construct map of pPEG-HSV1tk with the HSV1-tk encoding gene downstream of PEG-Prom.

FIG. 2A-C. Cancer-specific PEG-Prom activity shown by bioluminescence imaging (BLI) in an experimental model of human melanoma metastasis (Mel). Images were obtained at 48 h after the intravenous (IV) delivery of pPEG-Luc/PEI polyplex. Each animal was imaged from four directions (V, ventral; L, left side; R, right side; D, dorsal views) in order to cover the entire body. Pseudo-color images from the two groups were adjusted to the same threshold. Bioluminescent signal was observed specifically in the melanoma metastasis model. A, Quantification of BLI signal intensity in the control group (Ctrl) and Mel group at 24 and 48 h after injection of pPEG-Luc/PEI polyplex. Regions of interest (ROIs) were drawn over the thoracic cavity of animals on every image acquired for all four positions. Quantified values are shown in Total Flux (photons per second, p/s). ***P<0.0001; B and C) CT scans and gross anatomical views of lung from one representative animal from the control group (B) and the melanoma metastasis group (C). Black arrows indicate metastatic nodules observed in the lung.

FIGS. 3A and B. Cancer-specific PEG-Prom activity shown by BLI in an experimental model of human breast cancer metastasis (BCa). BLI of one representative animal from the control group and the experimental breast cancer metastasis group. Images were acquired at 48 h after the IV delivery of pPEG-Luc/PEI polyplex. Each mouse was imaged from four directions (V, ventral; L, left side; R, right side; D, dorsal views). Pseudo-color images from the two groups were adjusted to the same threshold. A, Quantification of bioluminescent signal intensity measured in ROIs drawn over the thoracic cavity of the animals, acquired from each orientation. Quantified intensity was expressed in Total Flux (p/s). **P=0.0066. B, a CT image and a macroscopic view of lung from a representative metastasis model of human breast cancer. Black arrows indicate metastatic nodules observed in the lung.

FIGS. 4A and B. Intergroup comparison of the gene delivery efficiency to lungs. After 48 h BLI session, the absolute amount of pPEG-Luc in lung tissues of each animal was quantified by quantitative real time PCR. A, Standard curve plot of CT value versus log ng pDNA (pPEG-Luc). B, Absolute quantification of the pDNA delivery efficiency to lungs using the standard curve method. While no significant difference was observed between the control and the experimental models of human melanoma metastasis (Mel), the breast cancer metastasis models (BCa) had significantly lower transfection efficiency compared to the control. Error bars represent means±s.e.m. (n=3 for Ctrl; n=3 for Mel; n=4 for BCa) (NS, no significant difference; *p=0.0345)

FIGS. 5 A and B. Comparison of constitutive CMV promoter activity in the healthy control (Ctrl) and experimental melanoma metastasis (Mel) groups. A, Serial BLI of one representative animal from the Ctrl and Mel groups. The images were acquired at 8, 24 and 45 h after the systemic delivery of pCMV-Tri/PEI polyplex. The animal model and pDNA/PEI polyplex were generated as described in Methods. Pseudo-color images of the two groups were adjusted to the same threshold values. B, Quantification of bioluminescent signal intensity measured in ROIs drawn over the thoracic cavity of the animals. No significant difference in the CMV promoter activity was observed between the Ctrl and Mel groups at any time points. Error bars represent means±s.e.m. (n=3 for Ctrl; n=3 for Mel)

FIG. 6A-C. Cancer-specific expression of HSV1-tk driven by PEG-Prom shown by SPECT-CT imaging in an experimental model of human melanoma metastasis (Mel). A and C, CT, SPECT and co-registered [¹²⁵I]FIAU SPECT-CT images of lungs in the healthy control group (A, n=3; Ctrl-1-3) and in the metastasis model of melanoma (C, n=5; Mel-1-5). Images were acquired at 48 h after IV injection of [¹²⁵I]FIAU, which was 94 h after IV administration of pPEG-HSV1tk/PEI polyplex. B, Quantification of lung SPECT images in A and C. ROIs of the same size and shape were drawn in the right lobes of the lung of each animal. Quantified radioactivity was expressed as Mean % ID/g (mean percent injected dose per gram of tissue). **P=0.0070.

FIG. 7A-D. Detection and localization of metastatic masses of melanoma after the systemic administration of pPEG-HSV1tk by SPECT-CT imaging. Transverse, coronal and sagittal views of co-registered SPECT-CT images of Mel-2 (A) and Mel-3 (B, C and D) from FIG. 6C. All images were obtained at 24 h after [¹²⁵I]FIAU injection, which was 70 h after the IV administration of pPEG-HSV1tk/PEI polyplex. Gross anatomical details of the metastatic masses that were located based on the SPECT-CT images in A, B, C and D. Multiple metastatic sites were detected by SPECT-CT imaging in Mel-2 (A, dotted circle). Necropsy of the corresponding area revealed melanoma masses under the brown adipose tissue in the upper dorsal area. (B) Accumulated radioactivity was detected adjacent to the thoracic mid-spine toward the left side (white arrow), which corresponded to a tumor mass at this location. Additional metastatic sites demonstrated by SPECT-CT imaging are shown in C and D (white arrow and dotted circle). Melanoma was uncovered immediately above the diaphragm (f, white dotted circle) and in the left inguinal lymph node, correlating with C and D. Cross-comparison of the PEG-Prom-mediated imaging and FDG-PET in a breast cancer metastasis model, BCa-1. Two nodules (Tu-1 and -2) were detected by [¹²⁵I]FIAU-SPECT near the heart and were confirmed by necropsy. While Tu-1 was detected by both methods, Tu-2, a smaller nodule attached to the heart, was not obvious in the PET image. SPECT images were acquired 48 h post-injection of [¹²⁵I]FIAU, which was 94 h after the pPEG-HSV1tk/PEI delivery. The PET images were acquired on the same day as the SPECT data.

FIGS. 8A and B. Evaluation of pDNA transfection efficiency to bone and brain through the in vivo jetPEI™-mediated systemic delivery. (a,b) Absolute quantitation of the amount of pDNA delivered to bone and brain by using quantitative real time PCR using the standard curve. 24 h and 48 h after the systemic delivery of pPEG-Luc/PEI polyplex into female NCR nu/nu mice (Charles River), bone marrow, femurs, knee and hip joints and brains were collected along with lungs as a positive control, and total DNA was extracted from the fresh unfrozen tissues. The absolute amount of pPEG-Luc delivered into each organ was quantified in ng pDNA (a) and in the pDNA copy number (b) per 100 ng total DNA. Error bars represent means±s.e.m. (n=3 per each time point)*Femurs: After the removal of bone marrow from the femur, only the femoral cortical bones were used for total DNA extraction.

FIG. 10A-E. PEG-PROM promoter. A, 2.0 kb PEG-3 promoter (SEQ ID NO: 1); B, exemplary minimal promoter (SEQ ID NO: 2); C, PEA3 protein binding sequence; D, TATA sequence; E, AP1 protein binding sequence.

FIGS. 11A and B. Cancer-specific AEG-Prom and PEG-Prom activity shown by BLI in PCa cell lines. (A) Human PCa cell lines PC3-ML, LNCaP, DU-145 and the normal counterpart, prostatic epithelial cells (PrEC), were transiently transfected with pAEG-Luc and pPEG-Luc. The indicated cells were transfected with Luc under control of the experimental promoters AEG-Prom, PEG-Prom, and a promoter-less empty vector (control) as a pDNA-PEI polyplex. These results are from independent experiments so direct comparison between the activities of the two promoters is not possible. However, the data indicates that both promoters show cancer-selective activity. (B) PC3-ML cells transfected with each pAEG-Luc and mutant pAEG-mEbox1&2-Luc plasmids. Luminescence was normalized for transfection efficiency (by co-transfection with the pGL4.74-[hRluc/TK] vector, which expresses renilla luciferase). Luminescence was normalized for cell number (by μg total protein). Column heights signify mean±standard deviation (SD) for three independent experiments.

FIG. 12A-I. Comparison of AEG-Prom and PEG-Prom activity in an experimental model of metastatic human PCa (PC3-ML). Top panels (A-D): (A, C) Representative healthy control mice, Ctrl-1 and Ctrl-2 (n=4). (B, D) Representative tumor models, PCa-1 and PCa-2 (n=4), developed by IV administration of PC3-ML cells. (A, B) BLI at 48 h after delivery of the AEG-Prom-driven firefly luciferase construct (pAEG-Luc-PEI) in Ctrl-1 and PCa-1, respectively. (C, D) BLI at 48 h after delivery of the PEG-Prom-driven firefly luciferase construct (pPEG-Luc-PEI) in Ctrl-2 and PCa-2, respectively. Each mouse was imaged from four orientations (D, dorsal; V, ventral; L, left side; R, right side) with a scale in photons/sec/cm²/steradian. Pseudocolor images from the four groups were adjusted to the same threshold. Bottom panels (E-H): Histological analysis of the photon-emitting regions in pAEG-Luc-PEI- and pPEG-Luc-PEI-treated mice that received IV PC3-ML cells (PCa-1 and PCa-2) and controls (Ctrl-1 and Ctrl-2). Microscopic lesions can be visualized with hematoxylin and eosin (H & E) (left) and immunohistochemistry of Luc expression (dark stain, right) in tumors in the lung (F, H) but not in the lungs from Ctrl-1 or Ctrl-2 (E, G). (I) Total BLI photon flux emanating from the lung in the Ctrl and PC3-ML groups at 24 h and 48 h after injection of pAEG-Luc-PEI and pPEG-Luc-PEI polyplexes. The difference between photon emission from PC3-ML and Ctrl groups was significant (*P<0.0001). Scale: 100 μm (H & E) and 2 mm (macroscopic views).

FIGS. 13A and B. Correlation between AEG-Prom-driven Luc expression and metastatic sites shown by histological analysis in an experimental model of human PCa (PC3-ML). Histological analysis of the photon-emitting regions in A, pAEG-Luc-PEI and B, pPEG-Luc-PEI treated PC3-ML mice (PCa-1 and PCa-2) and controls (Ctrl-1 and Ctrl-2) as displayed in FIG. 2. G, glomerulus; T, tumor, N, necrosis. Microscopic lesions can be visualized with H & E (left) and immunohistochemistry of Luc expression (dark stain, right) in tumors in the lung (B, F), right kidney (C, G), and liver (D, H) but not in the lung from Ctrl-1 and Ctrl-2 (A, E) or the necrotic liver regions (D, H—as indicated by the letter N).

FIG. 14A-C. Correlation between AEG-Prom-driven Luc expression and c-Myc expression shown by histological analysis in mouse PCa-1. Histological analysis of the photon-emitting regions in pAEG-Luc-PEI treated PC3-ML mouse (PCa-1) and control (Ctrl-1) as shown above in FIG. 12. Microscopic lesions can be visualized with H & E (left) and immunohistochemistry of Luc expression (dark stain, middle) and immunohistochemistry of c-Myc expression (dark stain, right) in tumors in the lung (B) and right kidney (C) but not in the lung from Ctrl-1 (A).

FIG. 15A-C. AEG-Prom-driven Luc detects tibial lesion in a model of human PCa metastatic to bone (PC3-ML). 5×10⁴ cells were inoculated into the left cardiac ventricle to achieve hematogenous spread, providing skeletal metastasis. BLI experiments were conducted at seven weeks after inoculation with tumor cells. (A, B) BLI at 48 h after delivery of pAEG-Luc-PEI in a representative healthy control, Ctrl-1 (A), and the PC3-ML model, PCa-4 (B). (C) Histological analysis confirmed tumor metastasis (T) next to the bone marrow (BM) in the left tibia, but not in the right tibia of PCa-4. Scale: 100 μm (H & E) and 2 mm (macroscopic views).

FIG. 16A-E. Luc expression in human PCa (PC3-ML) models is due to cancer-specific AEG-Prom activity rather than to differences in transfection efficiency between normal and malignant tissue. (A, B) BLI showing Luc expression in a representative healthy control Ctrl-1 (A) (n=4) and a PCa model, PCa-2 (B) (n=4). The images were acquired at 48 h after the intravenous delivery of pAEG-Luc-PEI polyplex. (C-D) Ex vivo BLI, gross pathology and whole body images (with boxes) reveal the source of signal. The dissected organs were imaged within five min following euthanasia. H & E staining confirmed the extensive metastasis in (C) lung, (D) liver, the right adrenal and the right renal cortex (not shown). Luciferase IHC of consecutive sections showed the correlative luciferase expression (dark stain). (E) Comparison of Luc plasmid delivery to high tumor burden and low tumor burden areas in liver and lungs of the PCa group (n=3, PCa-1-3) and liver and lung sections of control (n=3, Ctrl-1-3). The absolute amount of pAEG-Luc in lung and liver tissues of each animal was quantified by quantitative real-time PCR. The differences in transfection efficiency between areas of high tumor burden vs. those of low tumor burden within liver in same animal (*P<0.0005), as well as between areas of high tumor burden within liver vs. normal liver (**P=0.0078), were significant. No significant difference was observed in transfection efficiency in the lungs and liver tissue between the control group and the PCa group. Error bars represent mean±standard deviation (SD).

FIG. 17A-E. AEG-Prom-based SPECT/CT imaging detects distant metastasis not identified by NaF- or FDG-PET/CT. The experimental metastases model, PCa-3 (n=5), was developed by intra-cardiac injection of 5×10⁴ PC3-ML-Luc cells. SPECT/CT images for PCa-3 and a representative healthy control Ctrl-1 were obtained at 18-20 h after [¹²⁵I]FIAU injection, which was 66-68 h after IV administration of pAEG-HSV1tk-PEI polyplex. (A, B) NaF-PET/CT, AEG-Prom SPECT/CT and FDG-PET imaging in Ctrl-1 (A) and the PC3-ML-Luc model, PCa-3 (B). (D, E) Gross pathology of the metastatic nodules that were located on the basis of the SPECT/CT images. (C, D) Ex vivo BLI of the dissected organs, imaged within 20 min of euthanasia. (C-E) In PCa-3, pAEG-HSV1tk identified lesions across the right shoulder (L1), dorsal thoracic wall adjacent to mid spine (L3, black dotted circle) and the ventral midline of the sternum (L4, white dotted circle), and within the knee joints next to the bone marrow (L5, dotted circle), as confirmed by gross pathology. A possible L1 lesion was detected by FDG/PET (B). Color bar represents percentage injected dose per gram of tissue, (% ID/g).

FIG. 18. AEG-1 promoter (SEQ ID NO: 7).

### DETAILED DESCRIPTION

An embodiment of the invention provides nucleic acid constructs and methods for their use in cancer imaging, cancer treatment, and in methods which combine cancer imaging and treatment. Constructs designed for therapy generally comprise a cancer-specific or cancer selective promoter and a recombinant gene that encodes a therapeutic agent (e.g. a protein or polypeptide whose expression is detrimental to cancer cells, and/or or one which can stimulate the immune system to attack the cancer) operably linked to the cancer-specific promoter. Thus, targeted killing of cancer cells and/or immunoregulation occurs even when the constructs are administered systemically. Constructs designed for imaging comprise a cancer-specific/selective promoter and a recombinant gene that encodes a reporter molecule (and optionally, a complement of the reporter molecule) operably linked to the cancer-specific promoter. The reporter molecule is either detectable in its own right, and hence when it is expressed in a cancer cell renders the cancer cell detectable; or the reporter is capable of associating or interacting with a “complement” (e.g. a substrate) that is detectable or becomes detectable due to the interaction. Constructs designed for both imaging and therapy contain both a recombinant gene that encodes a reporter molecule operably linked to a cancer-specific/selective promoter and a therapeutic agent operably linked to the same copy of a cancer-specific/selective promoter, or to a second copy of the same cancer-specific/selective promoter, or to a different type of cancer-specific/selective promoter. Because the reporter is expressed only in cancer cells, the constructs encoding a reporter and the complement of the reporter can be safely administered systemically: even though both are distributed widely throughout the body of a subject, the complement encounters and interacts with the reporter only within cancer cells. In some applications, direct injection into a tumor could also be employed. In some embodiments, the reporter-complement association results in both imaging potential and lethality to the cancer cells. These constructs and methods, and various combinations and permutations thereof, are discussed in detail below.

The constructs described herein are highly effective for imaging and treating spontaneous metastasis.

### Promoters

The constructs of the invention include at least one transcribable element (e.g. a gene composed of sequences of nucleic acids) that is operably connected or linked to a promoter that specifically or selectively drives transcription within cancer cells. Expression of the transcribable element may be inducible or constitutive. Suitable cancer selective/specific promoters (and or promoter/enhancer sequences) that may be used include but are not limited to: PEG-PROM, astrocyte elevated gene 1 (AEG-1) promoter (AEG-Prom), survivin-Prom, human telomerase reverse transcriptase (hTERT)-Prom, hypoxia-inducible promoter (HIF-1-alpha), DNA damage inducible promoters (e.g. GADD promoters), metastasis-associated promoters (metalloproteinase, collagenase, etc.), ceruloplasmin promoter (Lee et al., Cancer Res Mar. 1, 2004 64; 1788), mucin-1 promoters such as DF3/MUC1 (see U.S. Pat. No. 7,247,297), HexII promoter as described in US patent application 2001/00111128; prostate-specific antigen enhancer/promoter (Rodriguez et al. Cancer Res., 57: 2559-2563, 1997); α-fetoprotein gene promoter (Hallenbeck et al. Hum. Gene Ther., 10: 1721-1733, 1999); the surfactant protein B gene promoter (Doronin et al. J. Virol., 75: 3314-3324, 2001); MUC1 promoter (Kurihara et al. J. Clin. Investig., 106: 763-771, 2000); H19 promoter as per U.S. Pat. No. 8,034,914; those described in issued U.S. Pat. Nos. 7,816,131, 6,897,024, 7,321,030, 7,364,727, and others; etc., as well as derivative forms thereof. Any promoter that is specific for driving gene expression only in cancer cells, or that is selective for driving gene expression in cancer cells, or at least in cells of a particular type of cancer (so as to treat and image e.g. prostate, colon, breast, etc. primary and metastatic cancer) may be used in the practice of the invention. By “specific for driving gene expression in cancer cells” we mean that the promoter, when operably linked to a gene, functions to promote transcription of the gene only when located within a cancerous, malignant cell, but not when located within normal, non-cancerous cells. By “selective for driving gene expression in cancer cells” we mean that the promoter, when operably linked to a gene, functions to promote transcription of the gene to a greater degree when located within a cancer cell, than when located within non-cancerous cells. For example, the promoter drives gene expression of the gene at least about 2-fold, or about 3-, 4-, 5-, 6-, 7-, 8-, 9-, or 10-fold, or even about 20-, 30-, 40-, 50-, 60-, 70-, 80-, 90- or 100-fold or more (e.g. 500- or 1000-fold) when located within a cancerous cell than when located within a non-cancerous cell, when measured using standard gene expression measuring techniques that are known to those of skill in the art.

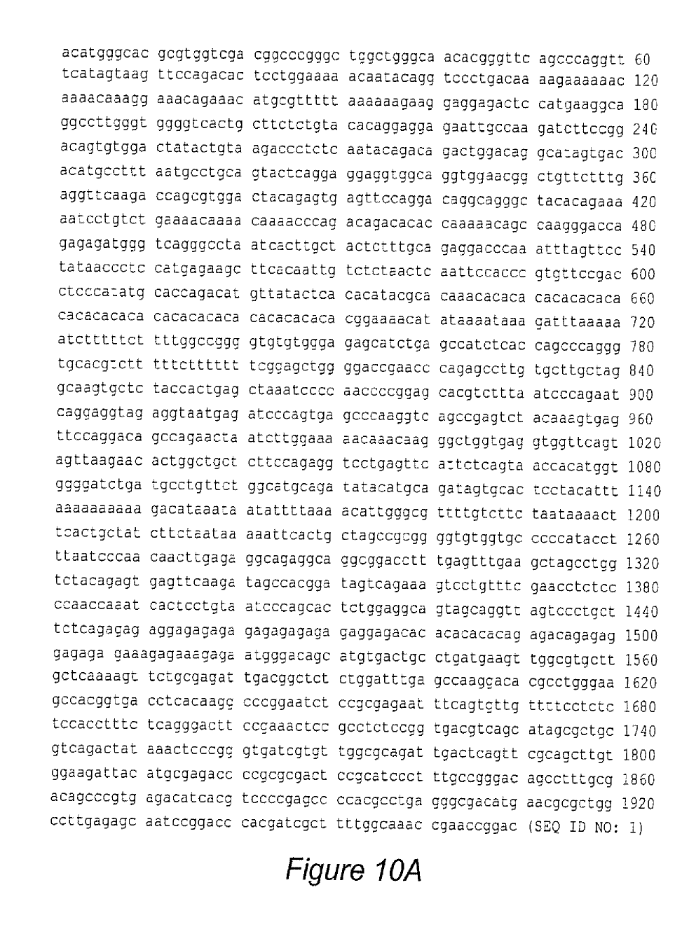

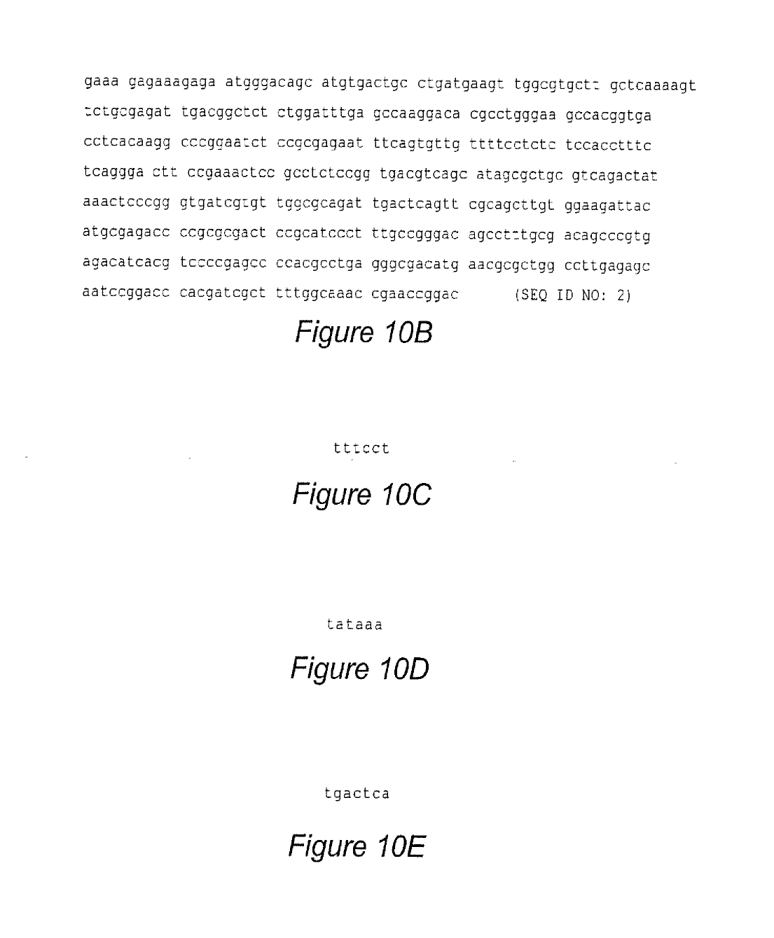

In one embodiment, the promoter is the PEG-PROM promoter (see FIG. 10A, SEQ ID NO: 1) or a functional derivative thereof. This promoter is described in detail, for example, in issued U.S. Pat. No. 6,737,523, the complete contents of which are herein incorporated by reference. In preferred embodiments, a “minimal” PEG-PROM promoter is utilized, i.e. a minimal promoter that includes a PEA3 protein binding nucleotide sequence (FIG. 10C, nucleotides 1507-1970 of SEQ ID NO: 1), a TATA sequence (e.g. FIG. 10D, nucleotides 1672-1677 of SEQ ID NO: 1), and an AP1 protein binding nucleotide sequence (FIG. 10E, nucleotides 1748-1753 of SEQ ID NO: 1), for example, the sequence depicted in FIG. 10B (SEQ ID NO:2), as described in U.S. Pat. No. 6,737,523. Nucleotide sequences which display homology to the PEG-PROM promoter and the minimal PEG-PROM promoter sequences are also encompassed for use, e.g. those which are at least about 50, 60, 70, 75, 80, 85, 90, 95, 96, 97, 98, or 99% homologous, as determined by standard nucleotide sequence comparison programs which are known in the art.

### Vectors

Vectors which comprise the constructs described herein are also encompassed by embodiments of the invention and include both viral and non-viral vectors. Exemplary non-viral vectors that may be employed include but are not limited to, for example: cosmids or plasmids; and, particularly for cloning large nucleic acid molecules, bacterial artificial chromosome vectors (BACs) and yeast artificial chromosome vectors (YACs); as well as liposomes (including targeted liposomes); cationic polymers; ligand-conjugated lipoplexes; polymer-DNA complexes; poly-L-lysine-molossin-DNA complexes; chitosan-DNA nanoparticles; polyethylenimine (PEI, e.g. branched PEI)-DNA complexes; various nanoparticles and/or nanoshells such as multifunctional nanoparticles, metallic nanoparticles or shells (e.g. positively, negatively or neutral charged gold particles, cadmium selenide, etc.); ultrasound-mediated microbubble delivery systems; various dendrimers (e.g. polyphenylene and poly(amidoamine)-based dendrimers; etc.

In addition, viral vectors may be employed. Exemplary viral vectors include but are not limited to: bacteriophages, various baculoviruses, retroviruses, and the like. Those of skill in the art are familiar with viral vectors that are used in “gene therapy” applications, which include but are not limited to: Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997), as well as viruses that are replication-competent conditional to a cancer cell such as oncolytic herpes virus NV 1066 and vaccinia virus GLV-1h68, as described in United States patent application 2009/0311664. In particular, adenoviral vectors may be used, e.g. targeted viral vectors such as those described in published United States patent application 2008/0213220.

Those of skill in the art will recognize that the choice of a particular vector will depend on its precise usage. Typically, one would not use a vector that integrates into the host cell genome due to the risk of insertional mutagenesis, and one should design vectors so as to avoid or minimize the occurrence of recombination within a vector's nucleic acid sequence or between vectors.

Host cells which contain the constructs and vectors of the invention are also encompassed, e.g. in vitro cells such as cultured cells, or bacterial or insect cells which are used to store, generate or manipulate the vectors, and the like. The constructs and vectors may be produced using recombinant technology or by synthetic means.

### Imaging

### Imaging Constructs and Vectors

In some embodiments, the invention provides gene constructs for use in imaging of cancer cells and tumors. The constructs include at least one transcribable element that is either directly detectable using imaging technology, or which functions with one or more additional molecules in a manner that creates a signal that is detectable using imaging technology. The transcribable element is operably linked to a cancer selective/specific promoter as described above, and is generally referred to as a “reporter” molecule. Reporter molecules can cause production of a detectable signal in any of several ways: they may encode a protein or polypeptide that has the property of being detectable in its own right; they may encode a protein or polypeptide that interacts with a second substance and causes the second substance to be detectable; they may encode a protein or polypeptide that sequesters a detectable substance, thereby increasing its local concentration sufficiently to render the surrounding environment (e.g. a cancer cell) detectable. If the gene product of the reporter gene interacts with another substance to generate a detectable signal, the other substance is referred to herein as a “complement” of the reporter molecule.

Examples of reporter proteins or polypeptides that are detectable in their own right (directly detectable) include those which exhibit a detectable property when exposed to, for example, a particular wavelength or range of wavelengths of energy. Examples of this category of detectable proteins include but are not limited to: green fluorescent protein (GFP) and variants thereof, including mutants such as blue, cyan, and yellow fluorescent proteins; proteins which are engineered to emit in the near-infrared regions of the spectrum; proteins which are engineered to emit in the short-, mid-, long-, and far-infrared regions of the spectrum; etc. Those of skill in the art will recognize that such detectable proteins may or may not be suitable for use in humans, depending on the toxicity or immunogenicity of the reagents involved. However, this embodiment has applications in, for example, laboratory or research endeavors involving animals, cell culture, tissue culture, various ex vivo procedures, etc.

Another class of reporter proteins is those which function with a complement molecule. In this embodiment, a construct comprising a gene encoding a reporter molecule is administered systemically to a subject in need of imaging, and a molecule that is a complement of the reporter is also administered systemically to the subject, before, after or together with the construct. If administered prior to or after administration of the construct, administration of the two may be timed so that the diffusion of each entity into cells, including the targeted cancer cells, occurs in a manner that results in sufficient concentrations of each within cancer cells to produce a detectable signal, e.g. typically within about 1 hour or less. If the two are administered “together”, then separate compositions may be administered at the same or nearly the same time (e.g. within about 30, 20, 15, 10, or 5 minutes or less), or a single composition comprising both the construct and the complement may be administered. In any case, no interaction between the reporter and the complement can occur outside of cancer cells, because the reporter is not produced and hence does not exist in any other location, since its transcription is controlled by a cancer specific/selective promoter.

One example of this embodiment is the oxidative enzyme luciferase and various modified forms thereof, the complement of which is luciferin. Briefly, catalysis of the oxidation of its complement, luciferin, by luciferase produces readily detectable amounts of light. Those of skill in the art will recognize that this system is not generally used in humans due to the need to administer the complement, luciferin to the subject. However, this embodiment is appropriate for use in animals, and in research endeavors involving cell culture, tissue culture, and various ex vivo procedures.

Another exemplary protein of this type is thymidine kinase (TK), e.g. TK from herpes simplex virus 1 (HSV 1), or from other sources. TK is a phosphotransferase enzyme (a kinase) that catalyzes the addition of a phosphate group from ATP to thymidine, thereby activating the thymidine for incorporation into nucleic acids, e.g. DNA. Various analogs of thymidine are also accepted as substrates by TK, and radiolabeled forms of thymidine or thymidine analogs may be used as the complement molecule to reporter protein TK. Without being bound by theory, it is believed that once phosphorylated by TK, the radiolabeled nucleotides are retained intracellularly because of the negatively charged phosphate group; or, alternatively, they may be incorporated into e.g. DNA in the cancer cell, and thus accumulate within the cancer cell. Either way, they provide a signal that is readily detectable and distinguishable from background radioactivity. Also, the substrate that is bound to TK at the time of imaging provides additional signal in the cancer cell. In fact, mutant TKs with very low Kms for substrates may augment this effect by capturing the substrate. The radioactivity emitted by the nucleotides is detectable using a variety of techniques, as described herein. This aspect of the use of TK harnesses the labeling potential of this enzyme; the toxic capabilities of TK are described below.

Various TK enzymes or modified or mutant forms thereof may be used in the practice of the invention, including but not limited to: HSV1-TK, HSV1-sr39TK, mutants with increased or decreased affinities for various substrates, temperature sensitive TK mutants, codon-optimized TK, the mutants described in U.S. Pat. No. 6,451,571 and US patent application 2011/0136221, both of which are herein incorporated by reference; various suitable human TKs and mutant human TKs, etc.

Detectable TK substrates that may be used include but are not limited to: thymidine analogs such as: “fialuridine” i.e. [1-(2-deoxy-2-fluoro-1-D -arabinofuranosyl)-5-iodouracil], also known as “FIAU” and various forms thereof, e.g. 2′-fluoro-2′-deoxy-β-D-5-[¹²⁵I]iodouracil-arabinofuranoside ([¹²⁵I]FIAU), [¹²⁴I]FIAU; thymidine analogs containing o-carboranylalkyl groups at the 3-position, as described by Al Mahoud et al., (Cancer Res Sep. 1, 2004 64; 6280), which may have a dual function in that they mediate cytotoxicity as well, as described below; hydroxymethyl]butyl)guanine (HBG) derivatives such as 9-(4-¹⁸F-fluoro-3-[hydroxymethyl]butyl)guanine (¹⁸F-FHBG); 2′-deoxy-2′-[¹⁸F]-fluoro-1-beta-D-arabinofuranosyl-5-iodouracil (¹⁸F-FEAU), 2′-deoxy-2′-[¹⁸F]-fluoro-5-methyl-1-β.-L-arabinofuranosyluracil (¹⁸F-FMAU), 1-(2′-deoxy-2′-fluoro-beta-D-arabinofuranosyl)-5-[¹⁸F]iodouracil (¹⁸F-FIAU), 2′-deoxy-2′-[¹⁸F]-fluoro-1-beta-D-arabinofuranosyl-5-iodouracil (¹⁸F-FIAC, see, for example, Chan et al., Nuclear Medicine and Biology 38 (2011) 987-995; and Cai et al., Nuclear Medicine and Biology 38 (2011) 659-666); various alkylated pyrimidine derivatives such as a C-6 alkylated pyrimidine derivative described by Muller et al. (Nuclear Medicine and Biology, 2011, in press); and others.

Other exemplary reporter molecules may retain or cause retention of a detectably labeled complement by any of a variety of mechanisms. For example, the reporter molecule may bind to the complement very strongly (e.g. irreversibly) and thus increase the local concentration of the complement within cancer cells; or the reporter molecule may modify the complement in a manner that makes egress of the complement from the cell difficult, or at least slow enough to result in a net delectable accumulation of complement within the cell; or the reporter may render the complement suitable for participation in one or more reactions which “trap” or secure the complement, or a modified form thereof that still includes the detectable label, within the cell, as is the case with the TK example presented above.

One example of such a system would be an enzyme-substrate complex, in which the reporter is usually the enzyme and the complement is usually the substrate, although this need not always be the case: the reporter may encode a polypeptide or peptide that is a substrate for an enzyme that functions as the “complement”. In some embodiments, the substrate is labeled with a detectable label (e.g. a radio-, fluorescent-, phosphoresent-, colorimetric-, light emitting-, or other label) and accumulates within cancer cells due to, for example, an irreversible binding reaction with the enzyme (i.e. it is a suicide substrate), or because it is released from the enzyme at a rate that is slow enough to result in a detectable accumulation within cancer cells, or the reaction with the enzyme causes a change in the properties of the substrate so that it cannot readily leave the cell, or leaves the cell very slowly (e.g. due to an increase in size, or a change in charge, hydrophobicity or hydrophilicity, etc.); or because, as a result of interaction or association with the enzyme, the substrate is modified and then engages in subsequent reactions which cause it (together with its detectable tag or label) to be retained in the cells, etc.

Other proteins that may function as reporter molecules in the practice of the invention are transporter molecules which are located on the cell surface or which are transmembrane proteins, e.g. ion pumps which transport various ions across cells membranes and into cells. An exemplary ion pup is the sodium-iodide symporter (NIS) also known as solute carrier family 5, member 5 (SLC5A5). In nature, this ion pump actively transports iodide (I⁻) across e.g. the basolateral membrane into thyroid epithelial cells, and the corresponding imaging agent [¹²⁴I]NaI can be detected using positron emission tomography (PET) scanning, or the corresponding imaging agent [¹²³I]NaI or [¹²⁵I]NaI can be detected using single photon emission computed tomography (SPECT). Recombinant forms of the transporter encoded by sequences of the constructs described herein may be selectively transcribed in cancer cells, and transport radiolabeled iodine into the cancer cells. Other examples of this family of transporters that may be used in the practice of the invention include but are not limited to norepinephrine transporter (NET); dopamine receptor, various estrogen receptor systems), ephrin proteins such as membrane-anchored ephrin-A (EFNA) and the transmembrane protein ephrin-B (EFNB); epidermal growth factor receptors (EGFRs); insulin-like growth factor receptors (e.g. IGF-1, IGF-2), etc.); transforming growth factor (TGF) receptors such as TGFα; etc. In these cases, the protein or a functional modified form thereof is expressed by the vector of the invention and the ligand molecule is administered to the patient. Usually, the ligand is labeled with a detectable label as described herein, or becomes detectable upon association or interaction with the transporter. In some embodiments, detection may require the association of a third entity with the ligand, e.g. a metal ion. The ligand may also be a protein, polypeptide or peptide.

In addition, antibodies may be utilized in the practice of the invention. For example, the vectors of the invention may be designed to express proteins, polypeptides, or peptides which are antigens or which comprise antigenic epitopes for which specific antibodies have been or can be produced. Exemplary antigens include but are not limited to tumor specific proteins that have an abnormal structure due to mutation (protooncogenes, tumor suppressors, the abnormal products of ras and p53 genes, etc.); various tumor-associated antigens such as proteins that are normally produced in very low quantities but whose production is dramatically increased in tumor cells (e.g. the enzyme tyrosinase, which is elevated in melanoma cells); various oncofetal antigens (e.g. alphafetoprotein (AFP) and carcinoembryonic antigen (CEA); abnormal proteins produced by cells infected with oncoviruses, e.g. EBV and HPV; various cell surface glycolipids and glycoproteins which have abnormal structures in tumor cells; etc. The antibodies, which may be monoclonal or polyclonal, are labeled with a detectable label and are administered to the patient after or together with the vector. The antibodies encounter and react with the expressed antigens or epitopes, which are produced only (or at least predominantly) in cancer cells, thereby labeling the cancer cells. Conversely, the antibody may be produced by the vector of the invention, and a labeled antigen may be administered to the patient. In this embodiment, an antibody or a fragment thereof, e.g. a Fab (fragment, antigen binding) segment, or others that are known to those of skill in the art, are employed. In this embodiment, the antigen or a substance containing antigens or epitopes for which the antibody is specific is labeled and administered to the subject being imaged.

Other examples of such systems include various ligand binding systems such as reporter proteins/polypeptides that bind ligands which can be imaged, examples of which include but are not limited to: proteins (e.g. metalloenzymes) that bind or chelate metals with a detectable signal; ferritin-based iron storage proteins such as that which is described by Iordanova et al, (Engineered Mitochondrial Ferritin as a Magnetic Resonance Imaging Reporter in Mouse Olfactory Epithelium. 2013. PLoS ONE 8(8): e72720. doi:10.1371/journal.pone.0072720), and others. Such systems of reporter and complement may be used in the practice of the invention, provided that the reporter or the complement can be transcribed under control of a cancer promoter, and that the other binding partner is detectable or can be detectably labeled, is administrable to a subject, and is capable of diffusion into cancer cells. Those of skill in the art will recognize that some such systems are suitable for use e.g. in human subjects, while others are not due to, for example, toxicity. However, systems in the latter category may be well-suited for use in laboratory settings.

In yet other aspects, the cancer-specific or cancer-selective promoters in the vectors of the invention drive expression of a secreted protein that is not normally found in the circulation. In this embodiment, the presence of the protein may be detected by standard (even commercially available) methods with high sensitivity in serum or urine. In other words, the cancer cells that are detected are detected in a body fluid.

In yet other embodiments, the cancer-specific or cancer-selective promoters in the vectors of the invention drive transcription of a protein or antigen to be expressed on the cell surface, which can then be tagged with a suitable detectable antibody or other affinity reagent. Candidate proteins for secretion and cell surface expression include but are not limited to: β-subunit of human chorionic gonadotropin (β hCG); human α-fetoprotein (AFP), and streptavidin (SA).

β hCG is expressed in pregnant women and promotes the maintenance of the corpus luteum during the beginning of pregnancy. The level of β hCG in non-pregnant normal women and men is 0-5 mIU/mL. hCG is secreted into the serum and urine and β hCG has been used for pregnancy test since the α-subunit of hCG is shared with other hormones. Urine β hCG can be easily detected by a chromatographic immunoassay (i.e. pregnancy test strip, detection threshold is 20-100 mIU/mL) at home- physician's office- and laboratory-based settings. The serum level can be measured by chemiluminescent or fluorescent immunoassays using 2-4 mL of venous blood for more quantitative detection. β hCG has been shown to secreted into the media when it was expressed in monkey cells. Human AFP is an oncofetal antigen that is expressed only during fetal development and in adults with certain types of cancers. AFP in adults can be found in hepatocellular carcinoma, testicular tumors and metastatic liver cancer. AFP can be detected in serum, plasma, or whole blood by chromatographic immunoassay and by enzyme immunoassay for the quantitative measurement.

Strepavadin (SA) can also be used as a cell surface target in the practice of the invention. The unusually high affinity of SA with biotin provides very efficient and powerful target for imaging and therapy. To bring SA to the plasma membrane of the cancer cells, SA can be fused to glycosylphosphatidylinositol (GPI)-anchored signal of human CD14. GPI-anchoring of SA will be suitable for therapeutic applications since GPI-anchor proteins can be endocytosed to the recycling endosomes. Once expressed on the cell surface, SA can then be bound by avidin conjugates that contain a toxic or radiotoxic warhead. Toxic proteins and venoms such as ricin, abrin, Pseudomonas exotoxin (PE, such as PE37, PE38, and PE40), diphtheria toxin (DT), saporin, restrictocin, cholera toxin, gelonin, Shigella toxin, and pokeweed antiviral protein, Bordetella pertussis adenylate cyclase toxin, or modified toxins thereof, or other toxic agents that directly or indirectly inhibit cell growth or kill cells may be linked to avidin; as could toxic low molecular weight species, such as doxorubicin or taxol or radionuclides such as ¹²⁵I, ¹³¹I, ¹¹¹In, ¹⁷⁷Lu, ²¹¹At, ²²⁵Ac, ²¹³Bi and ⁹⁰Y; antiangiogenic agents such as thalidomide, angiostatin, antisense molecules, COX-2 inhibitors, integrin antagonists, endostatin, thrombospondin-1, and interferon alpha, vitaxin, celecoxib, rofecoxib; as well as chemotherapeutic agents such as: pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers; caspase activators; and chromatin disruptors, especially those which can be conjugated to nanoparticles

### Detection of the Imaging Signal

The detectable components of the system (usually a complement or substrate) used in the imaging embodiment of the invention may be labeled with any of a variety of detectable labels, examples of which are described above. In addition, especially useful detectable labels are those which are highly sensitive and can be detected non-invasively, such as the isotopes ¹²⁵I, ¹²⁴I, ¹²³I, ⁹⁹mTc, ¹⁸F, ⁸⁶Y, ¹¹C, ¹²⁵I, ⁶⁴Cu, ⁶⁷Ga, ⁶⁸Ga, ²⁰¹Tl, ⁷⁶Br, ⁷⁵Br, ¹¹¹In, ⁸²Rb, ¹³N, and others.

Those of skill in the art will recognize that many different detection techniques exist which may be employed in the practice of the present invention, and that the selection of one particular technique over another generally depends on the type of signal that is produced and also the medium in which the signal is being detected, e.g. in the human body, in a laboratory animal, in cell or tissue culture, ex vivo, etc. For example, bioluminescence imaging (BLI); fluorescence imaging; magnetic resonance imaging [MRI, e.g. using lysine rich protein (LRp) as described by Gilad et al., Nature Biotechnology, 25, 2 (2007); or creatine kinase, tyrosinase, β-galactosidase, iron-based reporter genes such as transferrin, ferritin, and MagA; low-density lipoprotein receptor-related protein (LRP; polypeptides such as poly-L-lysine, poly-L-arginine and poly-L-threonine; and others as described, e.g. by Gilad et al., J. Nucl. Med. 2008; 49(12):1905-1908); computed tomography (CT); positron emission tomography (PET); single-photon emission computed tomography (SPECT); boron neutron capture; for metals:synchrotron X-ray fluorescence (SXRF) microscopy, secondary ion mass spectrometry (SIMS), and laser ablation inductively coupled plasma mass spectrometry (LA-ICP-MS) for imaging metals; photothermal imaging (using for example, magneto-plasmonic nanoparticles, etc.

### Therapy

Targeted cancer therapy is carried out by administering the constructs, vectors, etc. as described herein to a patient in need thereof. In this embodiment, a gene encoding a therapeutic molecule, e.g. a protein or polypeptide, which is deleterious to cancer cells is operably linked to a cancer-specific promoter as described herein in a “therapeutic construct” or “therapeutic vector”. The therapeutic protein may kill cancer cells (e.g. by initiating or causing apoptosis), or may slow their rate of growth (e.g. may slow their rate of proliferation), or may arrest their growth and development or otherwise damage the cancer cells in some manner, or may even render the cancer cells more sensitive to other anti-cancer agents, to immune recognition, etc.

Genes encoding therapeutic molecules that may be employed in the present invention include but are not limited to suicide genes, including genes encoding various enzymes; oncogenes; tumor suppressor genes; toxins; cytokines; oncostatins; TRAIL, etc. Exemplary enzymes include, for example, thymidine kinase (TK) and various derivatives thereof; TNF-related apoptosis-inducing ligand (TRAIL), xanthine-guanine phosphoribosyltransferase (GPT); cytosine deaminase (CD); hypoxanthine phosphoribosyl transferase (HPRT); etc. Exemplary tumor suppressor genes include neu, EGF, ras (including H, K, and N ras), p53, Retinoblastoma tumor suppressor gene (Rb), Wilm's Tumor Gene Product, Phosphotyrosine Phosphatase (PTPase), AdE1A and nm23. Suitable toxins include Pseudomonas exotoxin A and S; diphtheria toxin (DT); E. coli LT toxins, Shiga toxin, Shiga-like toxins (SLT-1, -2), ricin, abrin, supporin, gelonin, etc. Suitable cytokines include interferons and interleukins such as interleukin 1 (IL-1), IL-2, IL-3, IL-4, IL-5, IL-6, IL-7, IL-8, IL-9, IL-10, IL-1, IL-12, IL-13, IL-14, IL-15, IL-18, β-interferon, α-interferon, γ-interferon, angiostatin, thrombospondin, endostatin, GM-CSF, G-CSF, M-CSF, METH 1, METH 2, tumor necrosis factor, TGFβ, LT and combinations thereof. Other anti-tumor agents include: GM-CSF interleukins, tumor necrosis factor (TNF); interferon-beta and virus-induced human Mx proteins; TNFα and TNFβ; human melanoma differentiation-associated gene-7 (mda-7), also known as interleukin-24 (IL-24), various truncated versions of mda-7/IL-24 such as M4; siRNAs and shRNAs targeting important growth regulating or oncogenes which are required by or overexpressed in cancer cells; antibodies such as antibodies that are specific or selective for attacking cancer cells; etc.

When the therapeutic agent is TK (e.g. viral TK), a TK substrate such as acyclovir; ganciclovir, various thymidine analogs (e.g. those containing o-carboranylalkyl groups at the 3-position [Cancer Res Sep. 1, 2004 64; 6280]) is administered to the subject. These drugs act as prodrugs, which in themselves are not toxic, but are converted to toxic drugs by phosphorylation by viral TK. Both the TK gene and substrate must be used concurrently to be toxic to the host cancer cell.

### Imaging Plus Treatment

In some embodiments, the invention provides cancer treatment protocols in which imaging of cancer cells and tumors is combined with treating the disease, i.e. with killing, destroying, slowing the growth of, attenuating the ability to divide (reproduce), enhancing immune recognition or otherwise damaging the cancer cells. These protocols may be referred to herein as “theranostics” or “combined therapies” or “combination protocols”, or by similar terms and phrases.

In some embodiments, the combined therapy involves administering to a cancer patient a gene construct (e.g. a plasmid) that comprises, in a single construct, both a reporter gene (for imaging) and at least one therapeutic gene of interest (for treating the disease). In this embodiment, expression of either the reporter gene or the therapeutic gene, or preferably both is mediated by a cancer cell specific or selective promoter as described herein. Preferably, two different promoters are used in this embodiment in order to prevent or lessen the chance of crossover and recombination within the construct. Alternatively, tandem translation mechanisms may be employed, for example, the insertion of one or more internal ribosomal entry site (IRES) into the construct, which permits translation of multiple mRNA transcripts from a single mRNA. In this manner, both a reporter protein/polypeptide and a protein/polypeptide that is lethal or toxic to cancer cells are selectively or specifically produced within the targeted cancer cells.

Alternatively, the polypeptides encoded by the constructs of the invention (e.g. plasmids) may be genetically engineered to contain a contiguous sequence comprising two or more polypeptides of interest (e.g. a reporter and a toxic agent) with an intervening sequence that is cleavable within the cancer cell, e.g. a sequence that is enzymatically cleaved by intracellular proteases, or even that is susceptible to non-enzymatic hydrolytic cleavage mechanisms. In this case, cleavage of the intervening sequence results in production of functional polypeptides, i.e. polypeptides which are able to carry out their intended function, e.g. they are at least 50, 60, 70, 80, 90, or 100% (or possible more) as active as the protein sequences on which they are modeled or from which they are derived (e.g. a sequence that occurs in nature), when measured using standard techniques that are known to those of skill in the art.

In other embodiments of combined imaging and therapy, two different vectors may be administered, one of which is an “imaging vector or construct” as described herein, and the other of which is a “therapeutic vector or construct” as described herein.

In other embodiments of combined imaging and therapy, the genes of interest are encoded in the genome of a viral vector that is capable of transcription and/or translation of multiple mRNAs and/or the polypeptides or proteins they encode, by virtue of the properties inherent in the virus. In this embodiment, such viral vectors are genetically engineered to contain and express genes of interest (e.g. both a reporter gene and a therapeutic gene) under the principle control of one or more cancer specific promoters.

### Compositions

The present invention provides compositions, which comprise one or more vectors or constructs as described herein and a pharmacologically suitable carrier. The compositions are usually for systemic administration. The preparation of such compositions is known to those of skill in the art. Typically, they are prepared either as liquid solutions or suspensions, or as solid forms suitable for solution in, or suspension in, liquids prior to administration. The preparation may also be emulsified. The active ingredients may be mixed with excipients, which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any of one or more ingredients known in the art to provide the composition in a form suitable for administration. The final amount of vector in the formulations may vary. However, in general, the amount in the formulations will be from about 1-99%.

### Administration

The vector compositions (preparations) of the present invention are typically administered systemically, although this need not always be the case, as localized administration (e.g. intratumoral, or into an external orifice such as the vagina, the nasopharygeal region, the mouth; or into an internal cavity such as the thoracic cavity, the cranial cavity, the abdominal cavity, the spinal cavity, etc.) is not excluded. For systemic distribution of the vector, the preferred routes of administration include but are not limited to: intravenous, by injection, transdermal, via inhalation or intranasally, or via injection or intravenous administration of a cationic polymer-based vehicle (e.g. vivo-jetPEI™). Liposomal delivery, which when combined with targeting moieties will permit enhanced delivery. The ultrasound-targeted microbubble-destruction technique (UTMD) may also be used to deliver imaging and theranostic agents (Dash et al. Proc Natl Acad Sci USA. 2011 May 24; 108(21):8785-90. Epub 2011 May 9]; hydroxyapatite-chitosan nanocomposites (Venkatesan et al. Biomaterials. 2011 May, 32(15):3794-806); and others (Dash et al. Discov Med. 2011 January; 11(56):46-56. Review); etc. Any method that is known to those of skill in the art, and which is commensurate with the type of construct that is employed, may be utilized. In addition, the compositions may be administered in conjunction with other treatment modalities known in the art, such as various chemotherapeutic agents such a Pt drugs, substances that boost the immune system, antibiotic agents, and the like; or with other detections and imaging methods (e.g. to confirm or provide improved or more detailed imaging, e.g. in conjunction with mammograms, X-rays, Pap smears, prostate specific antigen (PSA) tests, etc.

Those of skill in the art will recognize that the amount of a construct or vector that is administered will vary from patient to patient, and possibly from administration to administration for the same patient, depending on a variety of factors, including but not limited to: weight, age, gender, overall state of health, the particular disease being treated, and other factors, and the amount and frequency of administration is best established by a health care professional such as a physician. Typically, optimal or effective tumor-inhibiting or tumor-killing amounts are established e.g. during animal trials and during standard clinical trials. Those of skill in the art are familiar with conversion of doses e.g. from a mouse to a human, which is generally done through body surface area, as described by Freireich et al. (Cancer Chemother Rep 1966; 50(4):219-244); and see Tables 1 and 2 below, which are taken from the website located at dtp,nci.nih.gov.

TABLE 1
Conversion factors in mg/kg

| | Mouse | Rat | Monkey | Dog | Human |
|---|---|---|---|---|---|
| | wt. 20 g | wt 150 g | wt 3 kg | wt 8 kg | wt 60 kg |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 1 2/3 | 1 | 1/2 |
| Man | 12 | 7 | 3 | 2 | 1 |

For example, given a dose of 50 mg/kg in the mouse, and appropriate does in a monkey would be 50 mg/kg×1/4=13 mg/kg/; or a dose of about 1.2 mg/kg is about 0.1 mg/kg for a human.

TABLE 2
Representative Surface Area to Weight Ratios

| Species | Body Weight (kg) | Surface Area (sq. m.) | Km factor |
|---|---|---|---|
| Mouse | 0.02 | 0.0066 | 3.0 |
| Rat | 0.15 | 0.025 | 5.9 |
| Monkey | 3.0 | 0.24 | 12 |
| Dog | 8.0 | 0.4 | 20 |
| Human, child | 20 | 0.8 | 25 |
| Human, adult | 60 | 1.6 | 37 |

To express the dose as the equivalent mg/sq.m. dose, multiply the dose by the appropriate factor. In adult humans, 100 mg/kg is equivalent to 100 mg/kg×37 kg/sq.m.=3700 mg/sq.m.

In general, for treatment methods, the amount of a vector such as a plasmid will be in the range of from about 0.01 to about 5 mg/kg or from about 0.05 to about 1 mg/kg (e.g. about 0.1 mg/kg), and from about 10⁵ to about 10²⁰ infectious units (IUs), or from about 10⁸ to about 10¹³ IUs for a viral-based vector. In general, for imaging methods, the amount of a vector will be in the range of from about 0.01 to about 5 mg/kg or from about 0.05 to about 1 mg/kg (e.g. about 0.1 mg/kg) of e.g. a plasmid, and from about 10⁵ to about 10²⁰ infectious units (IUs), or from about 10⁸ to about 10¹³ IUs for a viral-based vector. For combined imaging and therapy, the amounts of a vector will be in the ranges described above. Those of skill in the art are familiar with calculating or determining the level of an imaging signal that is required for adequate detection. For example, for radiopharmaceuticals such as [¹²⁴]FIAU, an injection on the order or from about 1 mCi to about 10 mCi, and usually about 5 mCi, (i.e. about 1 mg of material) is generally sufficient.

Further, one type of vector or more than one type of vector may be administered in a single administration, e.g. a therapy vector plus an imaging vector, or two (or more) different therapy vectors (e.g. each of which have differing modes of action so as to optimize or improve treatment outcomes), or two or more different imaging vectors, etc.

Typically cancer treatment requires repeated administrations of the compositions. For example, administration may be daily or every few days, (e.g. every 2, 3, 4, 5, or 6 days), or weekly, bi-weekly, or every 3-4 weeks, or monthly, or any combination of these, or alternating patterns of these. For example, a “round” of treatment (e.g. administration one a week for a month) may be followed by a period of no administration for a month, and then followed by a second round of weekly administration for a month, and so on, for any suitable time periods, as required to optimally treat the patient.

Imaging methods also may be carried out on a regular basis, especially when a subject is known or suspected to be at risk for developing cancer, due to e.g., the presence of a particular genetic mutation, family history, exposure to carcinogens, previous history of cancer, advanced age, etc. For example, annual, semi-annual, or bi-annual, or other periodic monitoring may be considered prudent for such individuals. Alternatively, individuals with no risk factors may simply wish to be monitored as part of routine health care, in order to rule out the disease.

For embodiments of the invention, which encompass both treatment and imaging, the administration protocols may be any which serve the best interest of the patient. For example, initially, an imaging vector alone may be administered in order to determine whether or not the subject does indeed have cancer, or to identify the locations of cancer cells in a patient that has already been diagnosed with cancer. Of note, the present method is very specific so that even very small masses of cancer cells can be visualized using the methods. If cancer is indeed indicated, then compositions with therapeutic vectors are then administered are needed to treat the disease. Usually a plurality of administrations is required as discussed above, and at least one, usually more, and sometimes all of these include at least one imaging vector together with a least one therapeutic vector; or optionally, a single vector with both capabilities. The ability to alternate between therapy and imaging, or to concomitantly carry out both, is a distinct boon for the field of cancer treatment. This methodology allows a medical professional to monitor the progress of treatment in a tightly controlled manner, and to adjust and/or modify the therapy as necessary for the benefit of the patient. For example, administration of a therapeutic and an imaging vector may be alternated; or, during early stages of treatment, initially an imaging vector may be administered, followed by therapy and imaging vectors together until the tumors are no longer visible, followed by imaging vector alone for a period of time deemed necessary to rule out or detect recurrence or latent disease.

The subjects or patients to whom the compositions of the invention are administered are typically mammals, frequently humans, but this need not always be the case. Veterinary applications are also contemplated.

Exemplary Types of Cancer that can be Treated

The constructs and methods of the invention are not specific for any one type of cancer. By “cancer” we mean malignant neoplasms in which cells divide and grow uncontrollably, forming malignant tumors, and invade nearby parts of the body. Cancer may also spread or metastasize to more distant parts of the body through the lymphatic system or bloodstream. The constructs and methods of the invention may be employed to image, diagnose, treat, monitor, etc. any type of cancer, tumor, neoplastic or tumor cells including but not limited to: osteosarcoma, ovarian carcinoma, breast carcinoma, melanoma, hepatocarcinoma, lung cancer, brain cancer, colorectal cancer, hematopoietic cell, prostate cancer, cervical carcinoma, retinoblastoma, esophageal carcinoma, bladder cancer, neuroblastoma, renal cancer, gastric cancer, pancreatic cancer, and others.

In addition, the invention may also be applied to imaging and therapy of benign tumors, which are generally recognized as not invading nearby tissue or metastasizing, for example, moles, uterine fibroids, etc.

### Transgenic Animals

The invention also encompasses transgenic non-human animals that have been genetically engineered to contain nucleotide sequences encoding a reporter gene operably linked to a cancer-specific or cancer-selective promoter, and their use for clinical evaluation of therapies. In the transgenic animals, the nucleotide sequences are stably integrated into the genome of the animal. In healthy animals, the promoter is not active and the reporter gene is not expressed. However, if such an animal develops cancer, then the promoter is induced or activated, and the reporter gene is expressed. Upon administration of the reporter complement to the animal, the development, location and fate of cancer cells can be monitored in detail. Such animals may be used for laboratory purposes, e.g. for testing carcinogenicity of substances, evaluating chemoprevention strategies and monitoring therapy. The animals can be exposed to potential carcinogens, administered complement, and then monitored to observe the effects of the potential carcinogen. Likewise, the effects of candidate anti-cancer agents can be tested or screened in the animals by administering the candidate either before attempting to induce cancer, or after cancer is established, and the effectiveness of the agent can be tracked and measured. Those of skill in the art are familiar with methods of evaluating the efficacy of drug candidates, including, for example, monitoring tumor location, stage, size, volume, appearance, frequency, duration, etc.

In other aspects, the PEG-PROM (or another cancer-specific or cancer-selective promoter) animals of the invention are further genetically altered to have a predisposition to the development of cancer. This may be done, for example, by cross breeding the animals with animals who already have the predisposition for cancer development (for example, any one of the number of mice that have been selected or genetically engineered to serve as model systems for various cancers). Alternatively, this may be accomplished by inducing desired genetic mutations in the cancer-specific or cancer-selective promoter, e.g. mutations which are associated with cancer development, or by further genetically engineering the animals to have a tendency to develop cancer.

Exemplary types of cancer-prone animals include any of those which are susceptible (or certain to develop) a cancer such as: breast cancer (e.g. mice such as mouse mammary tumor virus (MMTV)-neu transgenic mice; mouse MMTV-PyMT transgenic mice); prostate cancer (e.g. mice such as Hi-Myc, TRAMP, etc.); C3(1)/SV40 T antigen transgenic mouse model of prostate and mammary cancer; as well as animals which are models for melanoma, brain cancer, colorectal and intestinal cancer, etc. Such mice are available for example, from Jackson Labs in Bar Harbor, Me.

The animals that are genetically modified in this manner include but are not limited to: mice, rats, guinea pigs, rabbits, dogs, pigs, chickens, goats, primates such as marmosets, etc. Those of skill in the art are well acquainted with methods of genetically engineering and/or cross breeding and selecting animals for use in research.

### EXAMPLES

### Example 1

### Tumor-Specific Imaging through Progression Elevated Gene-3 Promoter-Driven Gene Expression

### Abstract

Molecular-genetic imaging is advancing from a valuable preclinical tool to guiding patient management. The strategy involves pairing an imaging reporter gene with a complementary imaging agent in a system that can be used to measure gene expression, protein interaction or track gene-tagged cells in vivo. Tissue-specific promoters can be used to delineate gene expression in certain tissues, particularly when coupled with an appropriate amplification mechanism. Here we show that the progression elevated gene-3 promoter (PEG-Prom), derived from a rodent gene mediating the malignant phenotype, can be used to drive imaging reporters selectively to enable detection of micrometastatic disease in murine models of human melanoma and breast cancer using bioluminescence and radionuclide-based molecular imaging techniques. Because of its strong promoter, tumor specificity and capacity for clinical translation, PEG-Prom-driven gene expression may represent a practical, new system by which to facilitate cancer imaging and imaging in combination with therapy.

### Introduction

A minimal promoter region of progression elevated gene-3 (PEG-3), a rodent gene, was previously identified for its association with malignant transformation and tumor progression using subtraction hybridization⁸. PEG-Prom drives downstream gene expression in a tumor-specific manner and has been tested in cancer cell lines of various tissues such as brain, prostate, breast and pancreas⁹⁻¹¹, as well as in metastatic melanoma¹². Transcription factors AP-1 and E1AF/PEA3 (ETS-1) are known to mediate the cancer-specific activity of PEG-Prom^{8,9,13}. Previous studies have demonstrated the utility of PEG-Prom for cancer gene therapy through intratumoral delivery^{9-12,14}. Here we describe a novel method for imaging a variety of metastatic cancers through systemic delivery of PEG-Prom. Based on these experiments it can be seen that the systemic delivery of PEG-Prom-driven imaging constructs will enable tumor-specific expression of reporter genes, not only within primary tumor, but also in associated metastases in a manner broadly applicable to tumors of different tissue origin or subtype.

### Methods

Additional detail regarding experimental procedures and results can be found above under “Brief Description of the Drawings”.
Plasmids. pPEG-Luc was constructed as described previously⁹. The Luc-encoding gene in pPEG-Luc was replaced by the HSV1-tk-encoding sequence from pORF-HSV1tk plasmid (InvivoGen) to generate pPEG-HSV1tk. pDNA were prepared with the EndoFree Plasmid Kit (Qiagen) and DNA pellets were dissolved in endotoxin-free water (Lonza). Endotoxin level was ensured as <2.5 endotoxin unit (EU)/mg pDNA with the ToxinSensor Gel Clot Endotoxin Assay Kit (GenScript).
Systemic DNA delivery. Low molecular weight 1-PEI-based cationic polymer, in vivo-jetPEI™, (Polyplus-transfection) provided the gene delivery vehicle. DNA-polyplex was formed according to the Manufacturer's Instructions. 30 μg of pDNA and 3.6 μl of 150 mM in vivo-jetPEI™ were diluted in endotoxin-free 5% glucose separately and then mixed together to give an N:P ratio of 6:1 in a total volume of 400 μl. The DNA-polymer mixture was incubated at room temperature for 15 min. 400 μl were injected into the lateral tail vein of an animal as two 200 μl-injections, within a 5 minute-interval.
Generation of experimental metastasis models. Animal studies were undertaken in accordance with the rules and regulations of the Johns Hopkins Animal Care and Use Committee. BLI studies employed experimental metastasis models of human melanoma (Mel) and breast cancer (BCa). 5-6 week-old female NCR nu/nu mice (NCI-Frederick) received whole body irradiation (5 Gy) to ensure suppression of the residual immune system in nude mice. Within 24 h after irradiation, animals were randomly divided into three groups. One group was injected with 5×10⁶ cells of the human malignant melanoma cell line MeWo (ATCC) intravenously (IV) to generate Mel. Another group of mice received IV injection of 2×106 cells of the human breast cancer cell line MDA-MB-231 for BCa. Another group was maintained as a control. In both models metastatic nodule formation in the lung was confirmed by CT at 4-7 weeks after cell injection. For the SPECT-CT studies the Mel model was generated as described above except that whole body irradiation was omitted. As a control group, we used female NCR nu/nu mice of the same age. MeWo and MDA-MB-231 cell lines were maintained in MEM and RPMI-1640 media, respectively, supplemented with 10% FBS and 1% penicillin/streptomycin.
In vivo bioluminescence imaging. At 24 and 48 h after gene delivery, animals were imaged with the IVIS Spectrum (Xenogen/Caliper). For each imaging session mice were injected intraperitoneally with D-luciferin (150 mg/kg) under anesthesia using 1.5-2.5% isoflurane/oxygen mixture. Images were acquired serially from 5-35 minutes after injection of D-luciferin. In order to compensate the limitation of 2D images, most animals were imaged in four different positions: ventral, left- and right-sided, dorsal. ROIs of the same size and shape, covering the entire thoracic cavity, were applied to the images to account for intra-group variations in metastatic site localization. Total Flux (p/s) in the ROIs was measured. One NCR nu/nu female mouse that did not receive any reagent was imaged with the same settings including binning and exposure time. The identical ROIs were applied to the images and the quantified total flux was used as background signal, which was subtracted from the measured counts from experimental animals. Image acquisition and BLI data analysis were done using Living Image softwares (Caliper Life Sciences).
SPECT-CT imaging and data analysis. At 46 h after injection of pPEG-HSV1tk/PEI polyplex, animals were injected intravenously with 51.8 mBq (1.4 mCi) of [¹²⁵I]FIAU. 24 and 48 h after radiotracer injection image data were acquired with the X-SPECT small-animal SPECT-CT system (Gamma Medica-Ideas, Inc.) using the low-energy single pinhole collimator (1.0 mm aperture). Focused lung imaging was acquired with a radius of rotation (ROR) of 3.35 cm and the whole body imaging with ROR of 6.75 cm. At 24 h after injection, animals were imaged in 64 projections with 5.625 degree increments and 30 sec of acquisition per projection, and at 48 h after injection with 60 sec per projection. SPECT images were co-registered with the 512-slice CT images. Tomographic image datasets were reconstructed with the 2D ordered subsets-expectation maximum (OS-EM) algorithm with two iterations and four subsets, and AMIDE38 and Amira (Visage Imaging) software was used for analysis.
PET-CT imaging and data analysis. At 1 h after 9.25 mBq (0.25 mCi) of IV administration of FDG, whole body images were acquired with the eXplore Vista small animal PET scanner (GE Healthcare) using the 250-700 keV energy window. Animals were fasted for 6-12 h prior to receiving FDG and were kept warm on the heating pad in order to minimize radiotracer accumulation in non-tumor tissues. PET images were co-registered with the 512-slice CT images. Tomographic image datasets were reconstructed with the 3D ordered subsets expectation maximization (OS-EM) algorithm with three iterations and twelve subsets and analyzed with AMIDE38 software.

Immunohistochemistry. After the BLI data acquisition at 48 h after the pPEG-Luc/PEI polyplex delivery, each organ demonstrating expression of Luc was harvested and fixed in 10% neutral buffered formalin. Paraffin-embedded 5 μm-thick slices and 25 μm-thick lung cryosections were stained with rabbit anti-luciferase polyclonal antibody (1:25 dilution of 50 μg/ml stock, Fitzgerald Industries International, Inc.) at room temperature for 1 h. Horseradish peroxidase (HRP)-conjugated polyclonal goat anti-rabbit antibody was used as a secondary antibody. HRP activity was detected with 3,3′-diaminobenzidine substrate-chromogen (EnVision™+Kit, Dako). Statistical analysis. Error bars in graphical data represent means±s.e.m. The two-tailed Student's t test was performed, with P<0.05 considered statistically significant.

### Results

### Cancer-Specific Activity of PEG-Prom Via Bioluminescence Imaging In Vivo

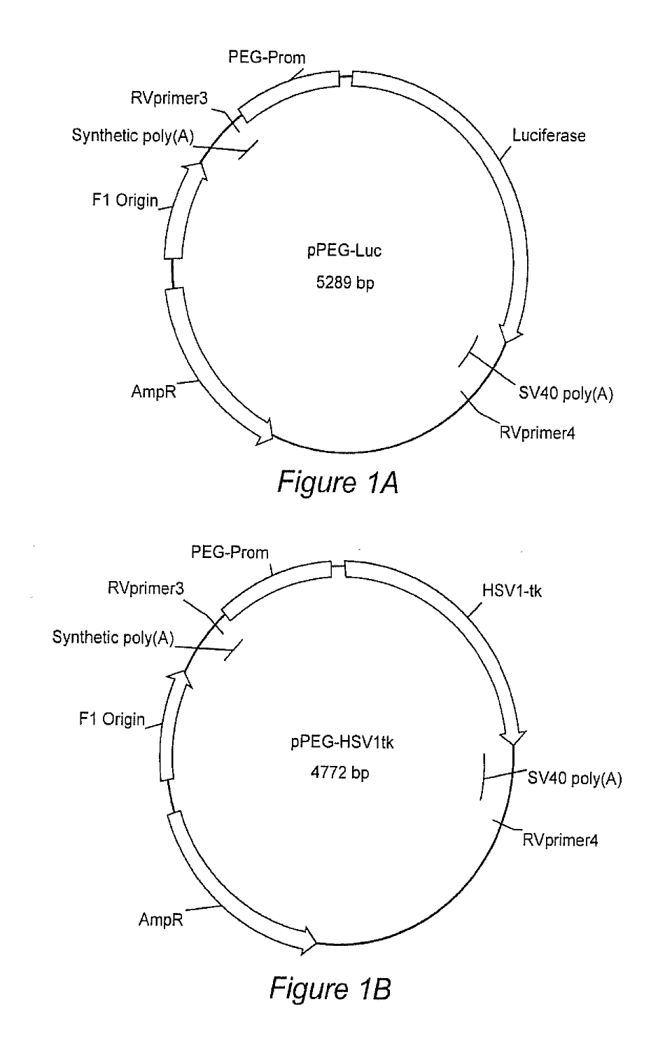

To test the specificity of PEG-Prom for tumor imaging in vivo, we used two different reporters, firefly luciferase (Luc) and the herpes simplex virus 1 thymidine kinase (HSV1-tk). Luc is often used with bioluminescence imaging (BLI) to establish proof-of-principle for imaging specific gene expression or gene-tagged cells in preclinical models, while HSV1-tk, also often used preclinically, has been translated to clinical studies. Accordingly, we generated two plasmid constructs, pPEG-Luc and pPEG-HSV1tk (FIG. 1). We chose to image the experimental metastasis models of two different tissues: human melanoma and breast cancer. As a gene delivery vehicle we used in vivo-jetPEI™, which is based on linear polyethylenimine (1-PEI), one of the most widely used cationic polymers for gene delivery. We chose that inert (nonviral) vehicle rather than a viral delivery system to avoid biased systemic delivery, as can be seen with viral vectors, which have a tendency to localize to liver upon intravenous (IV) administration^{15,16}.

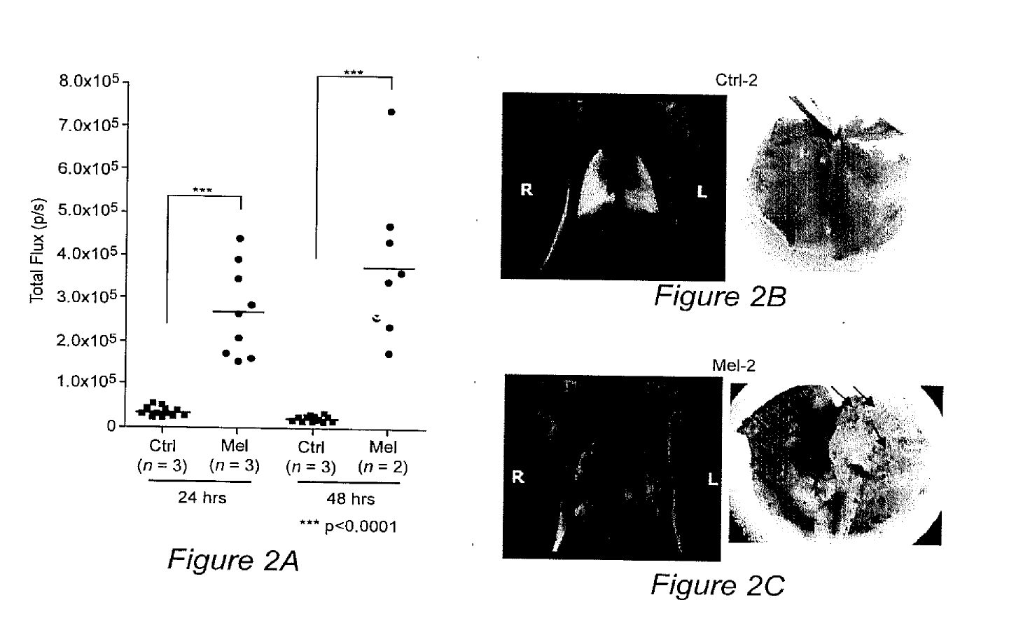

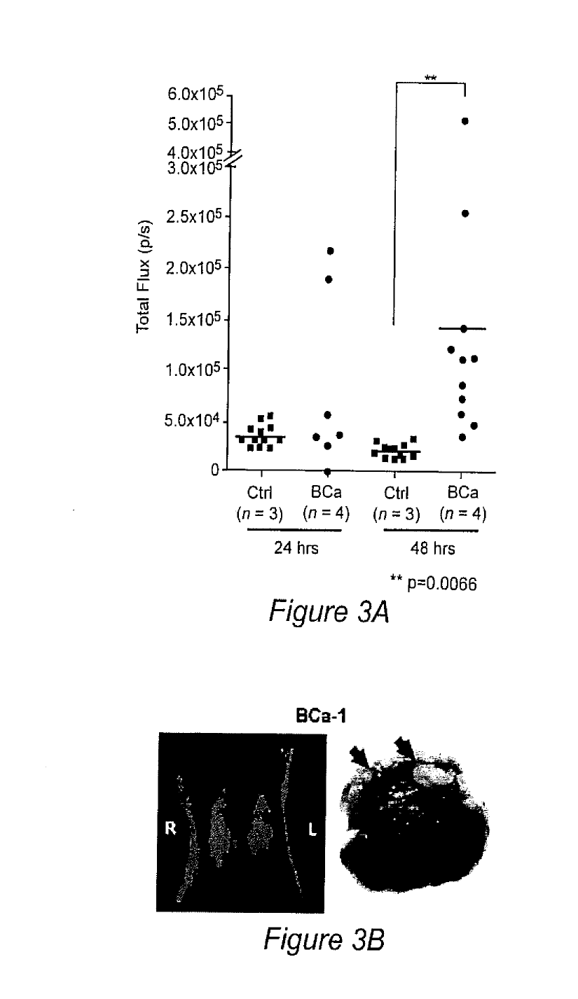

After confirmation of the presence of metastatic nodules in the lung by computed tomography (CT) at 4-6 weeks after IV administration of the human malignant melanoma cell line MeWo, or the human metastatic breast cancer cell line MDA-MB-231, animals received an IV dose of pPEG-Luc/PEI polyplex (FIG. 1A). Twenty four and forty eight hours after plasmid DNA (pDNA) delivery, PEG-Prom-driven gene expression was assessed by BLI. The same pDNA delivery and imaging protocols were applied to a group of healthy animals as a negative control. Expression of Luc driven by PEG-Prom was observed only in the melanoma metastasis model (Mel) and not in control animals (not shown). Control animals demonstrated nearly background levels of BLI output at the 24 h time point that disappeared by the 48 h imaging session (not shown). Quantification of the BLI signal intensity from the thoracic cavity, which represents Luc expression mainly in lung, shows significantly higher PEG-Prom activity in the Mel group compared to controls at both time points after pPEG-Luc administration (FIG. 2A), and more so at 48 h. Similar results were observed in the model of breast cancer metastasis (BCa) (FIGS. 3A and B). The same pseudo-color images of the control group were readjusted for the BCa model such that the control and BCa groups are scaled to the same threshold values. As with the Mel model, quantified bioluminescence intensity from the thoracic cavity shows higher PEG-Prom activity in the BCa group compared to controls, and more markedly so 48 h after pPEG-Luc delivery (FIG. 3A). It took longer for the BCa group harboring MDA-MB-231 metastases than for the Mel group with MeWo metastases to provide a significant increase in BLI signal over background, likely resulting from the lower efficiency of gene delivery in the BCa model, as discussed below. BLI Images of all of the animals in each group, Mel and BCa, as well as controls, at the same pseudo-color threshold values were obtained.

On average an approximately three-fold higher level of Luc expression was observed from the Mel group compared to the BCa group at 48 h. CT scans and gross anatomical views revealed very different patterns of metastatic nodule formation in the lung of those two models. While MeWo cells formed small nodules uniformly scattered throughout the lungs (FIG. 2C black arrows), MDA-MB-231 cells tended to form isolated large nodules (FIG. 3B, black arrows). Histological analysis using hematoxylin and eosin (H&E) staining of formalin-fixed paraffin-embedded (FFPE) lung sections demonstrated that metastases derived from MeWo cells in the Mel model were better vascularized (not shown), while necrotic centers were observed in the nodules formed in the lungs of BCa animals harboring metastases derived from MDA-MB-231 cells (not shown). In addition to decreasing the efficiency of gene delivery, the poor vascularization and consequent central necrosis of the BCa tumors may limit access of D-luciferin and oxygen to the tumor, which are necessary concomitants for productive BLI signal.

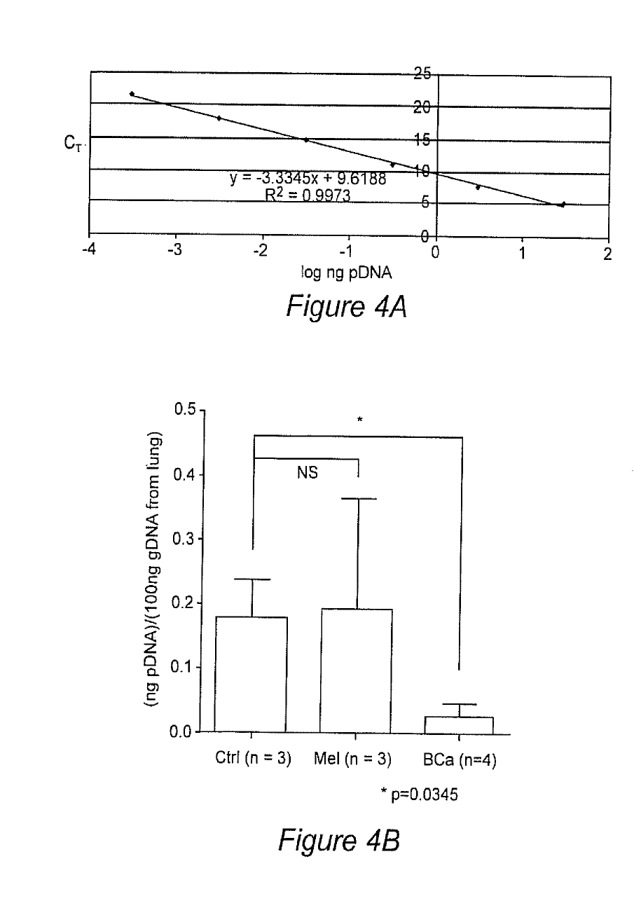

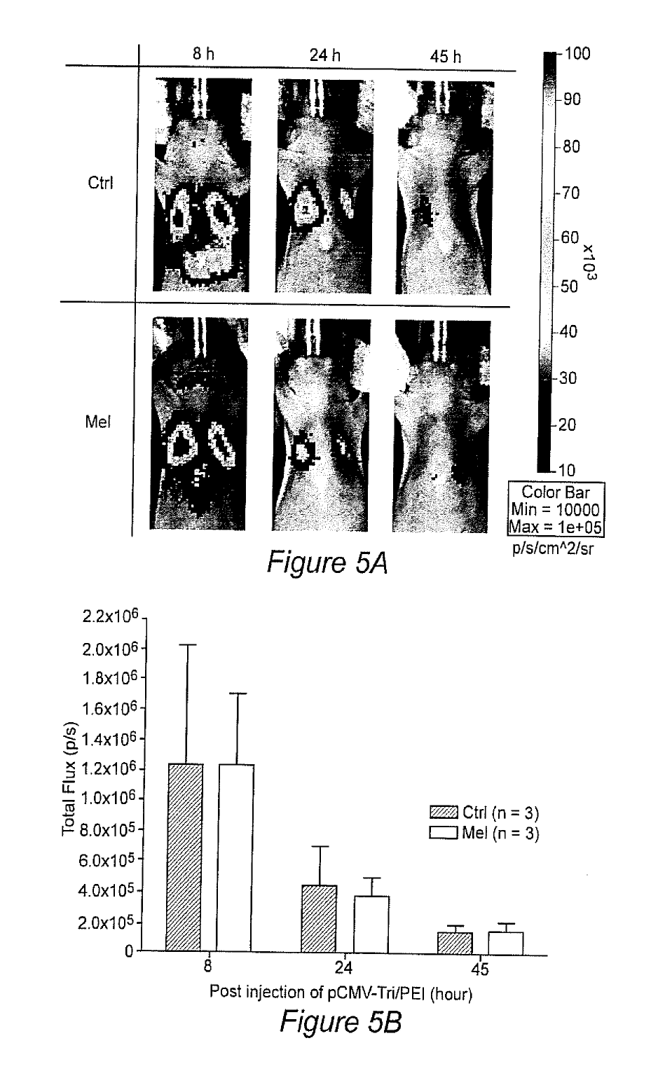

In order to exclude the possibility that tumor-specific expression of Luc by BLI might have resulted from the difference in transfection efficiency between normal and malignant mouse lung tissues, we quantified the amount of pDNA delivered to the lung of each animal. We performed quantitative real time PCR (qRT-PCR) using a primer set designed to amplify a region of the Luc-encoding gene in the pPEG-Luc plasmid. Total DNA extracted from the lung tissues was used as a template. The difference in transfection efficiency between the control group and the Mel group was not significant (FIGS. 4A and B). On the other hand, the BCa group had significantly lower transfection efficiency compared to the control. That result confirmed that the tumor-specific expression of Luc observed in these models was due to the tumor-selective activity of PEG-Prom rather than differential transfection efficiency between normal and malignant lungs. Poor vascularization and segregated large nodules most likely contributed to lower transfection efficiency observed in the lung of the BCa model. As a further check on the specific, PEG-Prom mediated nature of the aforementioned tumor imaging we also compared constitutive cytomegalovirus (CMV) promoter activity in the lungs of the healthy control and Mel groups (FIGS. 5A and B). BLI showed no significant difference in the CMV promoter-driven Luc expression level between the control and Mel groups at any time up to 45 h after the systemic delivery of pCMV-Tri/PEI polyplex. That result suggests that it is not a unique property of the tumor microenvironment, such as increased vascularity or enhanced permeability, causing greater plasmid expression in tumor relative to normal lung tissue.

BLI with systemically administered pPEG-Luc also enabled imaging of small metastatic deposits, i.e., micrometastases, outside of the lung parenchyma in both the Mel and BCa models. That was confirmed through harvesting regions producing BLI signal above background and performing correlative histological analysis. Specifically, histological analysis on the tissue sections from a representative Mel model, Mel-2, confirmed that Luc expression was associated with the metastatic sites formed in the lung, adrenal glands, the chest cavity adjacent to the sternum and abdominal inguinal adipose tissues adjoining the bladder. Similarly, correlation between metastatic sites and PEG-Prom activity was observed in a representative BCa model, BCa-3 inside the lung, the peripancreatic area, the thoracic wall adjacent to the sternum, a lymph node located in the adipose connective tissues surrounding the bladder and the rib cage in the form of thin rows of micrometastatic deposits.

### PEG-Prom-Mediated Cancer Detection Via Radionuclide Imaging In Vivo

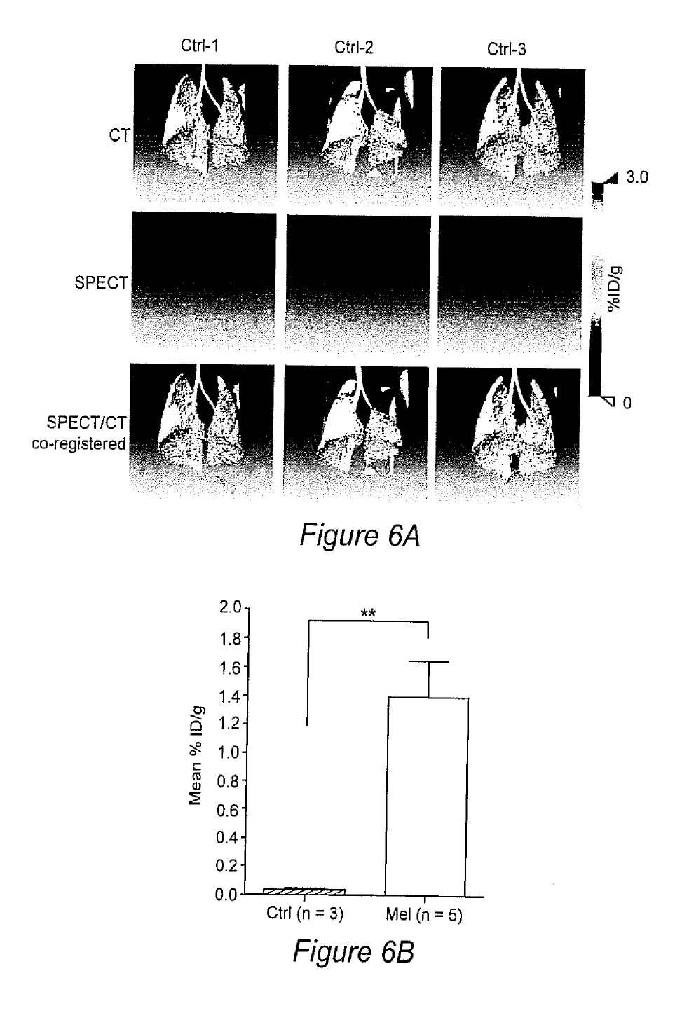

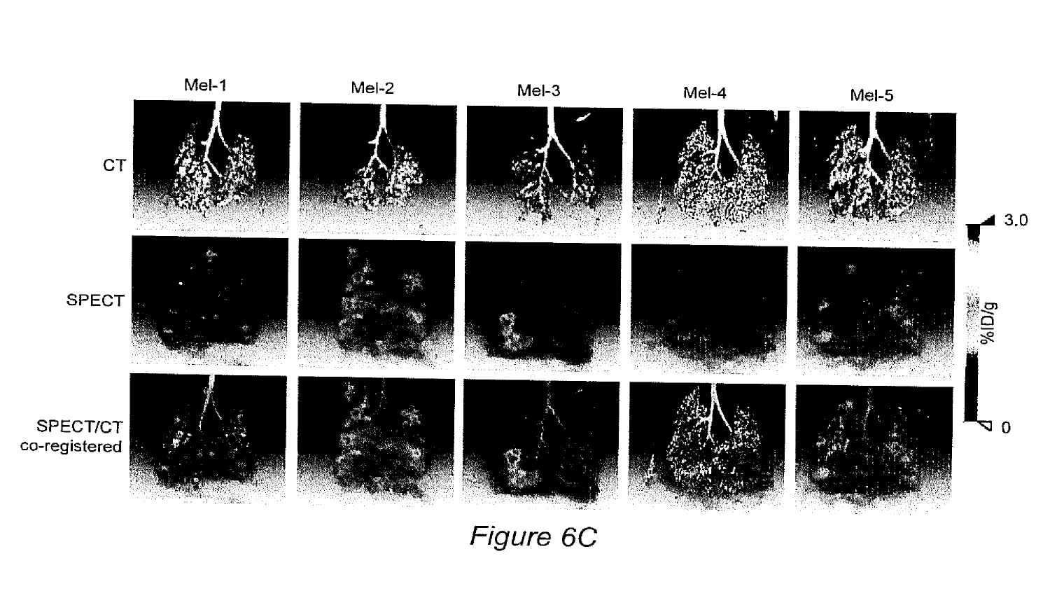

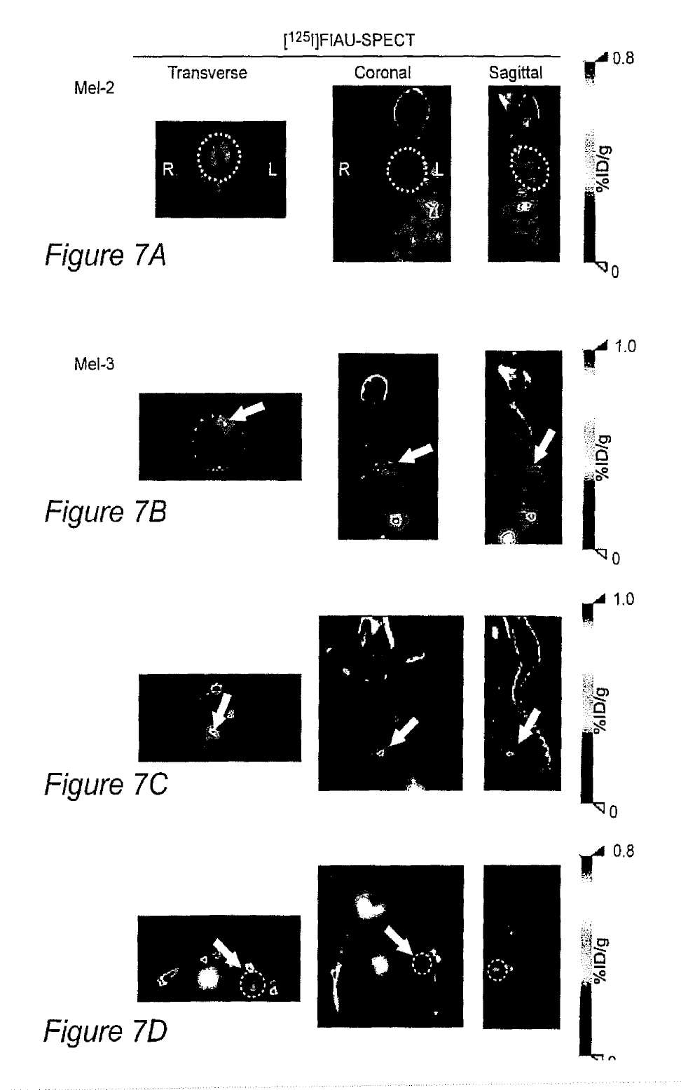

Although both malignant lung lesions and extrathoracic micrometastases could be detected with BLI, this technique is limited to preclinical studies. That is due to several factors, including the need to administer luciferase substrate, insufficient depth of penetration of BLI light output and difficulty in generating quantitative, tomographic BLI-based images. Accordingly, we generated a more clinically relevant PEG-Prom-driven gene expression imaging system, pPEG-HSV1tk (FIG. 1B), which can be detected using radionuclide-based techniques, namely, single photon emission computed tomography (SPECT) or positron emission tomography (PET), upon administration of a suitably radiolabeled nucleoside analog. We used the Mel experimental metastasis model to demonstrate tumor-targeted imaging with SPECT-CT. Approximately seven weeks after receiving MeWo cells as above, the Mel group and corresponding controls received pPEG-HSV1tk/PEI polyplex by IV injection. Forty six hours after pDNA delivery, the animals were injected with 2′-fluoro-2′-deoxy-β-D-5-[¹²⁵]iodouracil-arabinofuranoside ([¹²⁵I]FIAU) and imaged at 24 and 48 h after receiving the radiotracer (FIGS. 6A and C). Quantification of radioactivity demonstrates a 31-fold higher accumulation of [¹²⁵I]FIAU in the lungs of the Mel model compared to controls, indicating the tumor-specific expression of HSV1-tk under the control of PEG-Prom (FIG. 6B). We further confirmed tumor presence in presumptive extrathoracic metastatic sites through gross histological analysis after the 48 h imaging session. Detected on the whole body SPECT-CT images (FIG. 7A) were multiple metastatic lesions in the dorsal neck of Mel-2 that corresponded to the intact histological specimen. Metastatic sites, such as one to the left of the spinal cord, another immediately above the diaphragm and the other in the left inguinal lymph node, similarly correlated in Mel-3 (FIGS. 7 B-D). In order to evaluate the accuracy of detection and translational potential of PEG-Prom-mediated imaging, we compared the ability of the PEG-Prom system to detect lesions to that of [¹⁸F]fluorodeoxyglucose (FDG), the clinical standard. The same animals were imaged using each method. In most instances detected metastatic nodules correlated well between the two radionuclide-based techniques. However, the PEG-Prom-based system was better able to detect nodules adjacent to the heart and brown fat tissues, areas known to sequester FDG^{17,18}. That finding is particularly significant in light of the fact that SPECT is inherently at least one order of magnitude less sensitive than PET.

### Discussion

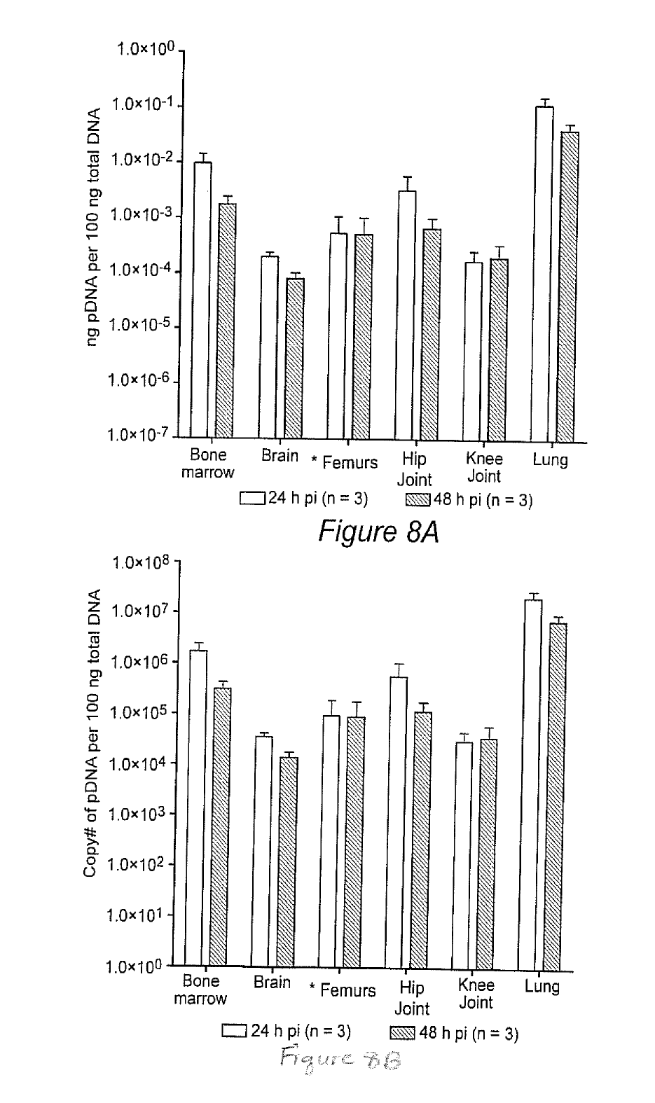

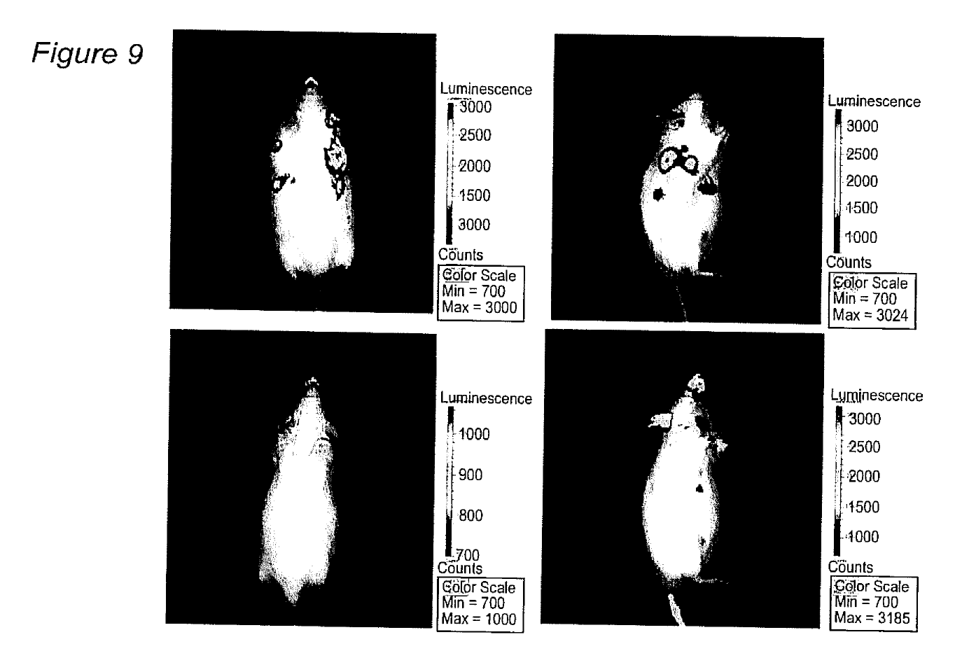

Our goal was to develop a systemically deliverable construct that would enable molecular-genetic imaging of cancer. Necessary elements to provide such a construct include a sufficiently strong promoter with cancer specificity, potential for clinical translation and capacity to be linked to gene therapy. Promoters derived from human telomerase reverse transcriptase (hTERT)4, survivin¹⁹ and carcinoembryonic antigen (CEA)²⁰ promoters and enhancer elements have been used in molecular-genetic imaging to provide tumor-specific reporter expression. However, because those studies employed adenoviral vectors, delivery was limited to local administration, systemic administration resulted in expression only within the liver. By contrast here we could delineate metastases with PEG-Prom after systemic delivery using a nonviral vector. Often promoter activity must be amplified to drive the downstream gene for purposes of imaging or therapy. One such strategy for doing so involves the two-step transcriptional amplification (TSTA) system^{21,22} using GAL4-VP16 fusion protein and the GAL4 response elements^{19,20,23-25}. However, PEG-Prom did not require amplification to achieve high-sensitivity imaging. SPECT-CT imaging demonstrated a metastatic to normal lung signal ratio of 31 out to four days after administration of pPEG-HSV1tk (FIG. 6B). PEG-Prom activity is comparable to the constitutively active SV40 promoter (data not shown). In keeping with previously reported in vitro results⁹, we demonstrate here that PEG-Prom proved to be tumor-specific in vivo using both imaging modalities and in both tumor models tested, with the potential for further generalization to other modalities and tumors. We further chose pPEG-HSV1tk because of its capacity to be translated clinically. Clinical molecular-genetic imaging and gene therapy have been accomplished using HSV1tk and radiolabeled nucleoside analogs^{7,26} and ganciclovir^{6,27-29}, respectively. By using the 1-PEI polyplex delivery vehicle we avoid the attendant problems of viral vectors in gene delivery, including immune reactions30 and oncogenesis. Using pDNA vectors the integration rate of the extrachromosomal gene into the host genome in vivo was negligible³¹⁻³⁴. We also estimated the potential of in vivo jetPEI™ as a pPEG-HSV1tk delivery vehicle for detection of bone and brain metastasis, which one may consider difficult for a nanoparticle delivery system to reach through systemic administration. Although lower than within lung, qRT-PCR demonstrated delivery of significant amounts of pDNA to each of those tissues (FIG. 8). Also difficult to reach would be necrotic areas within tumor, which are poorly vascularized. Molecular-genetic imaging techniques in general would be expected to have more efficient delivery of pDNA to the viable portions of tumor suggesting more accurate detection of well-vascularized as opposed to predominantly necrotic lesions.

Here we show how PEG-Prom can be used as an imaging agent for melanoma and breast cancer metastases in vivo and propose this promoter as potentially universal for this purpose. Such an agent could be used to detect tumors before their tissue of origin or subtype is identified, without concern for nonspecific expression in normal tissues. As with other imaging agents, PEG-Prom can be used not just for tumor detection, but also for preoperative planning, intraoperative management and therapeutic monitoring. The PEG-Prom imaging system can also be fashioned into a theranostic agent, through use of an internal ribosome entry site or other strategy enabling tandem gene expression. Promoters such as PSA (prostate-specific antigen) promoter^{23,24} for prostate cancer, mucin-1 promoter^{25,35} for breast cancer, and mesothelin promoter³⁶ for ovarian cancer have been used to delineate primary tumors and lymph node metastasis through molecular-genetic imaging. Similarly, although hTERT, survivin and CEA promoters were reported to be of a less tissue- and more cancer-specific nature, their activity relies on the transcription level of the marker genes. Rather, PEG-Prom is responsive directly to transcription factors unique to tumor cells. The PEG-3 gene is a truncated mutant form of the rat growth arrest- and DNA damage-inducible gene, GADD³⁴, which occurs uniquely during murine tumorigenesis and may function as a dominant-negative of GADD³⁴ promoting the malignant phenotype³⁷. No homolog to PEG-Prom is found in the human genome including the promoter/enhancer region of the human GADD homolog, which makes the use of PEG-Prom in human subjects likely to produce only minimal background signa^{19,37}.

These studies demonstrate that PEG-Prom may possess all of the necessary elements to provide a practical strategy for imaging and potentially image-guided therapy of a variety of cancers.

### REFERENCES FOR EXAMPLE 1

1. Blasberg, R. G. & Tjuvajev, J. G. Molecular-genetic imaging: current and future perspectives. J Clin Invest 111, 1620-1629 (2003).
2. Zhang, Y., et al. ABCG2/BCRP expression modulates D-Luciferin based bioluminescence imaging. Cancer Res 67, 9389-9397 (2007).
3. Uhrbom, L., Nerio, E. & Holland, E. C. Dissecting tumor maintenance requirements using bioluminescence imaging of cell proliferation in a mouse glioma model. Nat Med 10, 1257-1260 (2004).
4. Kishimoto, H., et al. In vivo imaging of lymph node metastasis with telomerase-specific replication-selective adenovirus. Nat Med 12, 1213-1219 (2006).
5. Padmanabhan, P., et al. Visualization of telomerase reverse transcriptase (hTERT) promoter activity using a trimodality fusion reporter construct. J Nucl Med 47, 270-277 (2006).
6. Freytag, S. O., et al. Phase I trial of replication-competent adenovirus-mediated suicide gene therapy combined with IMRT for prostate cancer. Mol Ther 15, 1016-1023 (2007).
7. Yaghoubi, S. S., et al. Noninvasive detection of therapeutic cytolytic T cells with 18F-FHBG PET in a patient with glioma. Nat Clin Pract Oncol 6, 53-58 (2009).
8. Su, Z. Z., Shi, Y. & Fisher, P. B. Subtraction hybridization identifies a transformation progression-associated gene PEG-3 with sequence homology to a growth arrest and DNA damage-inducible gene. Proc Natl Acad Sci USA 94, 9125-9130 (1997).
9. Su, Z. Z., et al. Targeting gene expression selectively in cancer cells by using the progression-elevated gene-3 promoter. Proc Natl Acad Sci USA 102, 1059-1064 (2005).
10. Sarkar, D., et al. Eradication of therapy-resistant human prostate tumors using a cancer terminator virus. Cancer Res 67, 5434-5442 (2007).
11. Sarkar, D., et al. Targeted virus replication plus immunotherapy eradicates primary and distant pancreatic tumors in nude mice. Cancer Res 65, 9056-9063 (2005).
12. Sarkar, D., et al. A cancer terminator virus eradicates both primary and distant human melanomas. Cancer Gene Ther 15, 293-302 (2008).
13. Su, Z., Shi, Y. & Fisher, P. B. Cooperation between AP1 and PEA3 sites within the progression elevated gene-3 (PEG-3) promoter regulate basal and differential expression of PEG-3 during progression of the oncogenic phenotype in transformed rat embryo cells. Oncogene 19, 3411-3421 (2000).
14. Sarkar, D., et al. Dual cancer-specific targeting strategy cures primary and distant breast carcinomas in nude mice. Proc Natl Acad Sci USA 102, 14034-14039 (2005).
15. Wood, M., et al. Biodistribution of an adenoviral vector carrying the luciferase reporter gene following intravesical or intravenous administration to a mouse. Cancer Gene Ther 6, 367-372 (1999).
16. Peng, K. W., et al. Organ distribution of gene expression after intravenous infusion of targeted and untargeted lentiviral vectors. Gene Ther 8, 1456-1463 (2001).
17. Evans, K. D., Tulloss, T. A. & Hall, N. 18FDG uptake in brown fat: potential for false positives. Radiol Technol 78, 361-366 (2007).
18. Shreve, P. D., Anzai, Y. & Wahl, R. L. Pitfalls in oncologic diagnosis with FDG PET imaging: physiologic and benign variants. Radiographics 19, 61-77; quiz 150-151 (1999).
19. Ray, S., et al. Noninvasive imaging of therapeutic gene expression using a bidirectional transcriptional amplification strategy. Mol Ther 16, 1848-1856 (2008).
20. Qiao, J., et al. Tumor-specific transcriptional targeting of suicide gene therapy. Gene Ther 9, 168-175 (2002).

21. Iyer, M., et al. Two-step transcriptional amplification as a method for imaging reporter gene expression using weak promoters. Proc Natl Acad Sci USA 98, 14595-14600 (2001).
22. Sadowski, I., Ma, J., Triezenberg, S. & Ptashne, M. GAL4-VP16 is an unusually potent transcriptional activator. Nature 335, 563-564 (1988).
23. Burton, J. B., et al. Adenovirus-mediated gene expression imaging to directly detect sentinel lymph node metastasis of prostate cancer. Nat Med 14, 882-888 (2008).
24. Iyer, M., et al. Noninvasive imaging of enhanced prostate-specific gene expression using a two-step transcriptional amplification-based lentivirus vector. Mol Ther 10, 545-552 (2004).
25. Huyn, S. T., et al. A potent, imaging adenoviral vector driven by the cancer-selective mucin-1 promoter that targets breast cancer metastasis. Clin Cancer Res 15, 3126-3134 (2009).
26. Jacobs, A., et al. Positron-emission tomography of vector-mediated gene expression in gene therapy for gliomas. Lancet 358, 727-729 (2001).
27. Immonen, A., et al. AdvHSV-tk gene therapy with intravenous ganciclovir improves survival in human malignant glioma: a randomised, controlled study. Mol Ther 10, 967-972 (2004).
28. Klatzmann, D., et al. A phase I/II study of herpes simplex virus type 1 thymidine kinase “suicide” gene therapy for recurrent glioblastoma. Study Group on Gene Therapy for Glioblastoma. Hum Gene Ther 9, 2595-2604 (1998).
29. Trask, T. W., et al. Phase I study of adenoviral delivery of the HSV-tk gene and ganciclovir administration in patients with current malignant brain tumors. Mol Ther 1, 195-203 (2000).
30. Bonnet, M. E., Erbacher, P. & Bolcato-Bellemin, A. L. Systemic delivery of DNA or siRNA mediated by linear polyethylenimine (L-PEI) does not induce an inflammatory response. Pharm Res 25, 2972-2982 (2008).
31. Coelho-Castelo, A. A., et al. Tissue distribution of a plasmid DNA encoding Hsp65 gene is dependent on the dose administered through intramuscular delivery. Genet Vaccines Ther 4, 1 (2006).
32. Kang, K. K., et al. Safety evaluation of GX-12, a new HIV therapeutic vaccine: investigation of integration into the host genome and expression in the reproductive organs. Intervirology 46, 270-276 (2003).
33. Manam, S., et al. Plasmid DNA vaccines: tissue distribution and effects of DNA sequence, adjuvants and delivery method on integration into host DNA. Intervirology 43, 273-281 (2000).
34. Ramirez, K., et al. Preclinical safety and biodistribution of Sindbis virus measles DNA vaccines administered as a single dose or followed by live attenuated measles vaccine in a heterologous prime-boost regimen. Hum Gene Ther 19, 522-531 (2008).
35. Dwyer, R. M., Bergert, E. R., O'Connor M, K., Gendler, S. J. & Morris, J. C. In vivo radioiodide imaging and treatment of breast cancer xenografts after MUC1-driven expression of the sodium iodide symporter. Clin Cancer Res 11, 1483-1489 (2005).
36. Tsuruta, Y., et al. A fiber-modified mesothelin promoter-based conditionally replicating adenovirus for treatment of ovarian cancer. Clin Cancer Res 14, 3582-3588 (2008).
37. Su, Z. Z., et al. Potential molecular mechanism for rodent tumorigenesis: mutational generation of Progression Elevated Gene-3 (PEG-3). Oncogene 24, 2247-2255 (2005).
38. Loening, A. M. & Gambhir, S. S. AMIDE: a free software tool for multimodality medical image analysis. Mol Imaging 2, 131-137 (2003).

### Example 2

### Overview

Targeted imaging of cancer remains a significant but elusive goal. Such imaging could provide early diagnosis, aid in treatment planning and profoundly benefit therapeutic monitoring. We identified the minimal promoter region of progression elevated gene-3 (PEG-Prom)^{1,2} derived from a rodent PEG-3 gene through subtraction hybridization³, whose expression directly correlates with malignant transformation and tumor progression in rodent tumors^{3,4}, as well as in human tumors, including cancer cell lines derived from tumors in the brain, prostate, breast, melanoma, and pancreas⁵⁻⁹. Based on these findings, we hypothesized and subsequently confirmed that systemic delivery of the PEG-Prom linked to and regulating an imaging construct would enable tumor-specific expression of reporter genes, not only within a primary tumor, but also in associated metastases in a manner broadly applicable to tumors of different tissue origin or subtype¹⁰. PEG-Prom is responsive directly to elevated transcription factors unique to tumor cells⁶⁻⁹, AP-1 and PEA-3, and no homolog has been found in the human genome, which makes the use of PEG-Prom in human subjects likely to produce only minimal background signal^{1,5}. Thus, the PEG-Prom can be used not just for tumor detection, but also for preoperative planning, intra-operative management and therapeutic monitoring.

Construction of a PEG-3-Luc mouse: Based on the transformation-specificity of the PEG-Prom, we developed a PEG-Luc transgenic mouse. To generate the PEG-3/luc2 transgene construct, a 446-bp fragment of the rat PEG-3 promoter (from −252 to +194) was inserted upstream of the rabbit β-globin region of pBS/pKCR3. The pBS/pKCR3 vector contains β-globin intron 2 and its flanking exons for efficient transgene expression¹¹. A PEG-3/β-globin composite fragment from the first construct was then inserted upstream of a synthetic firefly luciferase gene (luc2) in the pGL4.10[luc2] vector (Promega). To generate PEG-3/luc2 transgenic mice, a 3.4-kb SpeI/BamHI fragment was excised from the PEG-3/luc2 construct and evaluated for transgene expression. A PEG-3/β-globin composite fragment from the first construct was then inserted upstream of a synthetic firefly luciferase gene (luc2) in the pGL4.10[luc2] vector (Promega). To generate PEG-3/luc2 transgenic mice, a 3.4-kb SpeI/BamHI fragment was excised from the PEG-3/luc2 construct and microinjected into the male pronucleus of fertilized single-cell mouse embryos obtained from mating CB6F1 (C57BL/6×Balb/C) males and females. The injected embryos were then reimplanted into the oviducts of pseudopregnant CD-1 female mice. Offspring were screened for the presence of the PEG-3/luc2 transgene by PCR analysis of genomic tail DNA using a rabbit β-globin intron 2 sense primer (5′-CCCTCTGCTAACCATGTTCATGC-3′, SEQ ID NO: 3) and a luc2 antisense primer (5′-TCTTGCTCACGAATACGACGGTG-3′, SEQ ID NO: 4). Four potential founders carrying the PEG-3/luc2 transgene have been established and colonies of PEG-Luc mice have been developed.

Mouse mammary tumor virus (MMTV)-neu transgenic mice: Mouse mammary tumor virus (MMTV)-neu transgenic mice overexpresses NEU protein, the mouse homolog of the human her2 gene¹². This model carries an unactivated neu gene under the transcriptional control of the MMTV promoter/enhancer. Thus, the model simulates human her2-driven breast cancer by overexpression rather than point mutation of neu; resulting in focal mammary tumors and allowing for a realistic therapeutic study platform. MMTV-neu transgenic mouse develop focal mammary tumors during lactation and have a latency period of 7-8 months.
Development of double transgenic mice (MMTV-neu/PEG-Prom-Luc; MnPp-Luc) for in vivo imaging: Based on the cancer specific expression of the PEG-prom in human breast cancer cell lines, we hypothesized that the activity of the PEG-Prom will increase as mammary cells become transformed into tumors and metastases. To establish the proof-of-principal, we have generated MMTV-neu/PEG-Prom-Luc (MnPp-Luc) mice through mating between the MMTV-neu females with PEG-luc transgenic males from multiple PEG-luc lines to develop complex (MMTV-neu/PEG-Prom-Luc; MnPp-Luc) transgenic mice. As anticipated, the mammary tumor bearing mice (FIG. 9, Upper panel) expressed luciferase in confirmed tumors (by palpation and other areas in the mice), whereas the tumor negative mice had no significant luciferase expression in palpable tumors (FIG. 9, Lower panel). Based on these provocative findings, this complex transgenic animal model, which provides proof-of-principle for this strategy, will be useful to assay the efficacy of therapeutic and chemoprevention approaches at different stages of disease, including early stages and progression to metastasis, using non-invasive bioluminescence (BLI) approaches. We have collected different organs and are now investigating the histopathological correlations with BLI.

Of significance, this studies highlights the relevance of the Peg-Prom-Luc animal model in producing complex transgenic tumor animal models that can employ BLI for monitoring tumor development, progression to metastasis, and monitoring and evaluating various modes of therapeutic intervention (including treatment with cytotoxic, apoptosis-inducing, toxic autophagy-inducing and necrosis-inducting agents; viral therapeutic approaches; immune therapies, etc.). In addition, the PEG-Prom-Luc animals (or animals with other cancer-specific or cancer-selective promoters integrated into the germ line) could be used as single transgenic animals to look at processes such as skin carcinogenesis, organ carcinogenesis as a result of exposure to specific toxic agents and the role of chemoprevention in preventing or limiting the severity of cancer induction and progression.

In conclusion, these studies are paradigm shifting, providing proof-of-principle for developing cancer diagnostic mice (OncoView Mice). They further provide evidence for the utility of the PEG-Prom-Luc/double transgenic mouse approach for producing OncoView Mice in which cancer development and progression can be imaged using BLI. Moreover, this approach is not restricted to only breast cancer, since it can, in principle, be applied to any cancerous transgenic animal model including but not limited to pancreas, prostate, lung, colorectum, brain, ovary, esophagus, stomach, skin (melanoma) and others.

### REFERENCES FOR EXAMPLE 2

1. Su Z Z, Sarkar D, Emdad L, Duigou G J, Young C S H, Ware J, Randolph A, Valerie K, and Fisher P B. Targeting gene expression selectively in cancer cells by using the progression-elevated gene-3 promoter. Proc Natl Acad Sci USA 2005; 102(4):1059-1064.
2. Su Z, Shi Y, Fisher P B. Cooperation between AP1 and PEA3 sites within the progression elevated gene-3 (PEG-3) promoter regulate basal and differential expression of PEG-3 during progression of the oncogenic phenotype in transformed rat embryo cells. Oncogene 2000; 19(30):3411-21.
3. Su Z Z, Shi Y, Fisher P B. Subtraction hybridization identifies a transformation progression-associated gene PEG-3 with sequence homology to a growth arrest and DNA damage-inducible gene. Proc Natl Acad Sci USA 1997; 94(17):9125-30.
4. Su Z Z, Goldstein N I, Jiang H, Wang M N, Duigou G J, Young C S, Fisher P B. PEG-3, a nontransforming cancer progression gene, is a positive regulator of cancer aggressiveness and angiogenesis. Proc Natl Acad Sci USA 1999; 96(26):15115-20.
5. Su Z Z, Emdad L, Sarkar D, Randolph A, Valerie K, Yacoub A, Dent P, Fisher P B. Potential molecular mechanism for rodent tumorigenesis: mutational generation of Progression Elevated Gene-3 (PEG-3). Oncogene 2005; 24(13):2247-55.
6. Sarkar D, Su Z Z, Vozhilla N, Park E S, Gupta P, Fisher P B. Dual cancer-specific targeting strategy cures primary and distant breast carcinomas in nude mice. Proc Natl Acad Sci USA 2005; 102(39): 14034-9.
7. Sarkar D, Su Z Z, Vozhilla N, Park E S, Randolph A, Valerie K, Fisher, P B. Targeted virus replication plus immunotherapy eradicates primary and distant pancreatic tumors in nude mice. Cancer Res 2005; 65(19):9056-63.
8. Sarkar D, Lebedeva I V, Su Z Z, Park E S, Chatman L, Vozhilla N, Dent P, Curiel, D T, Fisher P B. Eradication of therapy-resistant human prostate tumors using a cancer terminator virus. Cancer Res 2007; 67(11):5434-5442.
9. Sarkar D, Su Z Z, Park E S, Vozhilla N, Dent P, Curiel D T, Fisher P B. A cancer terminator virus eradicates both primary and distant human melanomas. Cancer Gene Therapy 2008; 15(5):293-302.
10. Bhang H E C, Gabrielson K L, Laterra J, Fisher P B, Pomper M G. Tumor-specific imaging through progression elevated gene-3 promoter-driven gene expression. Nature Medicine 2011; 17(1):123-9.
11. Howes K A, Ransom N, Papermaster D S, Lasudry J G, Albert D M, Windle J J. Apoptosis or retinoblastoma: alternative fates of photoreceptors expressing the HPV-16 E7 gene in the presence or absence of p53. Genes Dev 1994; 8(11):1300-10.
12. Jolicoeur P, Bouchard L, Guimond A, Step-Marie M, Hanna Z, Dievart A. Use of mouse mammary tumour virus (MMTV)/neu transgenic mice to identify genes collaborating with the c-erbB-2 oncogene in mammary tumour development. Biochem Soc Symp. 1998; 63:159-65.

### Example 3

A transcription-based imaging, therapeutic or theranostic system can be considered for clinical translation if it meets certain criteria such as high tumor specificity, broad application and minimal toxicity (1). The first two criteria can be met through the choice of a strong and tumor-specific promoter. For example, cancer-specific gene therapy with the osteocalcin promoter, delivered through intra-lesional administration of an adenoviral vector, caused apoptosis in a subset of patients with prostate cancer (PCa) (2). We have previously shown that cancer-specific imaging could be accomplished in vitro and in vivo in experimental models by placing imaging reporters under the control of the progression elevated gene-3 promoter (PEG-Prom) (1, 3). Here we show that by employing the astrocyte elevated gene-1 promoter (AEG-Prom) (4) for cancer-specific imaging, focusing on metastatic models of PCa, for which there is no reliable clinical imaging agent.

AEG-1 was first identified using subtraction hybridization as an up-regulated gene in primary human fetal astrocytes infected with HIV-1 (5, 6). Subsequent studies identified AEG-1 as a metastasis-associated gene in the mouse, called metadherin (MTDH) (7), and as a lysine-rich CEACAM1 co-isolated gene in the rat, called LYRIC (8). Recent studies in multiple cancer indications confirm a significant role for AEG-1 as an oncogene (9) implicated in cancer development and progression in many organ sites (10). Based on the potentially diverse roles of AEG-1 in tumor progression, including transformation, growth regulation, cell survival, prevention of apoptosis, cell migration and invasion, metastasis, angiogenesis, and resistance to chemotherapy (11), this gene provides a viable target for therapies for diverse cancers. Expression of AEG-1 involves transcriptional regulation through defined sites in its promoter (4). A minimal promoter region of AEG-1 was identified by virtue of its association with oncogenic Ha-ras-induced transformation (4). AEG-1 is a downstream target of the Ha-ras and c-myc oncogenes, accounting in part for its tumor-specific expression. We have previously shown that AEG-Prom is activated by the binding of the transcription factors c-Myc and its partner Max to the two E-box elements of the promoter in Ha-ras-transformed rodent and immortalized transformed astrocyte cell lines (4). AEG-1 interacts with PLZF, the transcriptional repressor that regulates the expression of the genes involved in cell growth and apoptosis (12). AEG-Prom acts as a broadly applicable cancer sensor and has been tested in a spectrum of malignancies, including those involving brain, prostate, breast and pancreas, among others (P. B. Fisher, unpublished data).

Although molecular-genetic imaging with AEG-Prom should be generally applicable to a variety of malignancies, our initial study performed here was in part to demonstrate the utility of this system in a relevant and challenging application, namely, for molecular imaging of PCa. We also focus on PCa because positron emission tomography (PET) with [¹⁸F]fluorodeoxyglucose (FDG), which is the current clinical standard for a wide variety of malignancies, does not work particularly well for this disease (13). Although a variety of new molecular imaging agents for PET with computed tomography (PET/CT) of PCa are emerging, such as [¹⁸F]NaF (NaF) (14, 15), [¹¹C]- and [¹⁸F]choline (16-18), [¹⁸F]FDHT (19), anti-[¹⁸F]FACBC (20) and [¹⁸F]DCFBC (21), some are limited to detecting bone lesions (NaF), have significant overlap with normal prostate tissue (the cholines), or have not yet been extensively tested in the clinic. To maintain relevance to clinical translation, we used a linear polyethyleneimine (1-PEI) nanoparticle to deliver the construct systemically. Nanoparticles comprised of 1-PEI are being used in a variety of ongoing clinical trials (22-24). We describe AEG-Prom-mediated imaging in tumors derived from PC3-ML cells, a human androgen-independent invasive and metastatic model of PCa (25-27). We show that imaging with AEG-Prom delineates lesions from PCa as well as or with higher sensitivity than FDG- or NaF-PET/CT in this model system.

### Materials and Methods

Cloning of plasmid constructs. pPEG-Luc and pAEG-Luc were generated as described previously (3, 28). The firefly luciferase-encoding gene in pAEG-Luc was replaced by the HSV1-tk-encoding sequence from pORF-HSVtk plasmid (InvivoGen, San Diego, Calif.) to generate pAEG-HSV1tk. Details of cloning by restriction enzyme digestion and other conditions are available upon request. The plasmid DNA was purified with the EndoFree Plasmid Kit (Qiagen, Valencia, Calif.). Endotoxin level was ensured as <2.5 endotoxin units per mg of plasmid DNA.
Cell lines. PC3-ML-Luc (stable transfectants) and PC3-ML were kindly provided by Dr. Mauricio Reginato (Drexel University, Philadelphia, Pa.). These were routinely cultured in Dulbecco's modified Eagle's medium (DMEM) (Cellgro, Manassas, Va.) supplemented with 10% (vol/vol) FBS and 1% (vol/vol) antimycotic solution (Sigma-Aldrich, St. Louis, Mo.) and incubated at 37° C., 5% CO₂. PrEC (normal prostate epithelium) cells were kindly provided by Dr. John T. Isaacs (Johns Hopkins School of Medicine, Baltimore, Md.). Those were grown in keratinocyte serum-free medium (total [Ca²⁺] is 75±2 μmol/L) supplemented with bovine pituitary extract and recombinant epidermal growth factor (Invitrogen Life Technologies, Grand Island, N.Y.).

Transient transfection and luciferase assay. The following PCa cell lines: PC3-ML, LNCaP, DU145, PrEC (primary cells) were plated in 6-well plates (BD Biosciences, Bedford, Mass., USA) at 180×10³-200×10³ cells. Cells were transfected using in-vitro jetPRIME® (Polyplus-transfection, Illkrich, France) according to the manufacturer's instructions. The indicated cells were transfected with Luc under the experimental promoters AEG-Prom, PEG-Prom, and a promoter-less empty vector (control) as a pDNA-PEI polyplex. Luminescence was normalized for transfection efficiency by co-transfection with a vector expressing renilla luciferase, pGL4.74[hRluc/TK](Promega, Madison, Wis.). After 48 h of transfection, the expression level of the Luc reporter was measured by the Dual Luciferase Reporter Assay kit (Promega). Luminescence was normalized for cell number (by μg total protein) using the BCA Protein Assay Kit (Pierce Biotechnology, Rockford, Ill.).

Construction of mutant AEG-Prom. The mEbox1 and mEbox2 sites were mutated in the wild-type pAEG-Luc plasmid to generate the pAEG-mEbox1&2-Luc plasmid. The consensus E-box sequence, CACGTG, was mutated into AGAGTG using the QuikChange Lightening Site-Directed Mutagenesis Kit (Agilent Technologies, Santa Clara, Calif.) and appropriate primers. The sequences of the forward (F) and reverse (R) primers for mutagenesis were F: 5′ CTGGTCTACAGTAACGGGTCC (SEQ ID NO: 5) and R: 5′ ATTCAGCCCATATCGTTTC (SEQ ID NO: 6). The mutated constructs were confirmed by sequencing (Integrated DNA Technologies, Coralville, Iowa). PC3-ML cells were transiently transfected with the wild-type and mutated plasmid for the subsequent luciferase assay, as described above.
Generation of an in viva experimental model of metastatic PCa. All protocols involving the use of animals were approved by the Johns Hopkins Animal Care and Use Committee. Four-to-six-week old male NOG (NOD/Shi-scid/IL-2Rγ^{null}) mice were purchased from the Sidney Kimmel Comprehensive Cancer Center's Animal Resources Core (Johns Hopkins School of Medicine). PC3-ML and PC3-ML-Luc cells were expanded over three to five passages. The cells were harvested and diluted in sterile Dulbecco's PBS lacking Ca²⁺ and Mg²⁺ (Invitrogen Life Technologies). For intravenous injection, mice were administered 1×10⁶ PC3-ML cells in 100 μL of sterile Dulbecco's PBS via the tail vein. To ensure hematogenous dissemination, including to the bone, the cells were injected into the left ventricle of the heart (26, 27). For this intra-cardiac model mice were anesthetized with ketamine (100 mg/kg) and xylazine (20 mg/kg) and inoculated into the left ventricle with 5×10⁴ PC3-ML-Luc enriched or PC3-ML cells in a total volume of 100 μL of sterile Dulbecco's PBS using a 26^{3/4}-gauge needle. To image the PC3-ML-Luc cells with BLI, mice were injected intraperitoneally (IP) with 100 μL of 25 mg/mL of D-luciferin solution (Caliper LifeSciences, Hopkinton, Mass.), and BLI was performed 20 min after the intra-cardiac injection to detect the distribution of cells. Mice were imaged weekly. Images were acquired on an IVIS Spectrum small animal imaging system (Caliper Life Sciences, Alameda, Calif.) and results were analyzed using Living Image software (Caliper Life Sciences). A group of age-matched healthy NOG mice served as a negative control for the PCa metastasis model.
Enrichment of PC3-ML-Luc cells. The PC3-ML-Luc cells were further selected for bone-homing tendency. Mice bearing PC3-ML-Luc tumors developed through the intra-cardiac injection method were monitored for tumor formation by BLI. After five weeks, following euthanasia the femur and tibia of the regions demonstrating clear signal were aseptically dissected. The tumor cells were established in culture by mincing the epiphysis and flushing the bone marrow with 1×PBS (Invitrogen Life Technologies) as described previously(25). The subpopulations of cells selected using a Transwell migration chamber with an 8 μm pore size (BD Falcon, San Jose, Calif.) were tested and confirmed for Luc expression as described previously (29), but using 1 mM of D-luciferin, potassium salt (Gold Biotechnology, St. Louis, Mo.). The radionuclide imaging experiments were performed with the enriched PC3-ML-Luc cell lines.
Systemic delivery of plasmid constructs. Low molecular weight 1-PEI-based cationic polymer, in vivo-jetPEI® (Polyplus Transfection), was used for gene delivery. The DNA-PEI polyplex was formed according to the manufacturer's instructions. For systemic delivery 40 μg of DNA and 4.2 μL of 150 mM in vivo-jetPEI® was diluted in endotoxin-free 5% (wt/vol) glucose separately. The glucose solutions of DNA and 1-PEI polymer were then mixed together to give an N:P ratio (the number of nitrogen residues of vivo-jetPEI® per number of phosphate groups of DNA) of 6:1 in a total volume of 400 μL. The DNA-PEI polyplex was injected IV as two 200 μL injections with a 5 min interval.
Bioluminescence imaging. In vivo BLI was conducted at 24 and 48 h after the systemic delivery of reporter genes. Mice were imaged with the IVIS Spectrum. For each imaging session mice were injected IP with 150 mg/kg of D-luciferin, potassium salt under anesthesia using a 2.0% isoflurane/oxygen mixture. Ex vivo BLI was conducted within 10 min of necropsy. Living Image 2.5 and Living Image 3.1 software were used for image acquisition and analysis.
SPECT-CT imaging and data analysis. At 48 h after injection of pAEG-HSV1tk/PEI polyplex, animals were injected IV with 37.0 MBq (1.0 mCi) of [¹²⁵I]FIAU. At 18-20 h after radiotracer injection, imaging data were acquired with the X-SPECT small-animal SPECT-CT system (Gamma Medica Ideas, Northridge, Calif.) using the low-energy single pinhole collimator (1.0 mm aperture). Focused lung and liver imaging were acquired with a radius of rotation of 3.35 cm and whole-body imaging was undertaken with a radius of rotation of 7.00 cm. Mice were imaged in 64 projections at 45 sec of acquisition per projection. SPECT images were co-registered with the corresponding 512-slice CT images. Tomographic image datasets were reconstructed with the 2D ordered subsets-expectation maximum (OS-EM) algorithm. AMIDE (30) and PMOD (v3.3, PMOD Technologies Ltd, Zurich, Switzerland) software were used for image quantification and analysis.
FDG- and NaF-PET/CT imaging and analysis. 9.25 MBq (0.25 mCi) of each imaging agent was injected via the tail vein. Animals were placed on a heating pad and were allowed mobility during the 1 h radiotracer uptake period. The animals were then subjected to isoflurane anesthesia. Whole-body images were acquired with the eXplore Vista small animal PET scanner (GE Healthcare, Milwaukee, Wis.) using the 250-700 keV energy window. Acquisition time was 30 min (two bed positions, 15 min per bed position). Mice were fasted for 6-12 h before receiving FDG to minimize radiotracer accumulation in non-tumor tissues. FDG and NaF imaging was done between four and five weeks after injection of PC3-ML-Luc cells. PET images were co-registered with the corresponding 512-slice CT images. Tomographic image datasets were reconstructed with the 3D OS-EM algorithm with three iterations and 12 subsets and were analyzed with AMIDE software (30).
Histological analysis. After BLI data acquisition at 48 h after pAEG-Luc-PEI delivery, each organ demonstrating expression of Luc was collected and fixed in 10% neutral buffered formalin. Tissues were embedded in paraffin blocks. Serial paraffin longitudinal sections were stained with goat anti-luciferase polyclonal antibody (Promega) or rabbit anti-Myc polyclonal antibody (Epitomics, Burlingame, Calif.). Horseradish peroxidase (HRP)-conjugated polyclonal rabbit anti-goat antibody was used as a secondary antibody. HRP activity was detected with 3,3′-diaminobenzidine (DAB) substrate chromogen (EnVision™+Kit, Dako, Carpinteria, Calif.). Consecutive sections of each tissue sample were stained with hematoxylin and eosin (H & E) and were photographed with a Zeiss photomicroscope III.
Quantitative real-time PCR. After imaging experiments, animals were euthanized and their lung and liver tissue were harvested and snap frozen. Total DNA was extracted by using DNeasy Blood & Tissue Kit (Qiagen) following the manufacturer's instructions. 100 ng of purified total DNA form each animal was used as a template. Quantitative real-time PCR was performed in triplicate per template using the inventoried Taqman® Gene Expression Assays (Cat. #4331182, Life Technologies, Grand Island, N.Y.) with the FAM dye labeled primer set for Luc. Reaction conditions were set as 50° C. for 2 min, 95° C. for 10 min and 50 cycles of 95° C. for 15 sec, 60° C. for 1 min followed by the disassociation step of 95° C. for 15 sec, 60° C. for 15 sec, 95° C. for 15 sec in a Bio-Rad iQ™5 Multicolor Real-Time PCR Detection system (Bio-Rad Laboratories, Hercules, Calif.). Data were analyzed by the absolute quantification method using a standard curve by iQ5 v2.0 software (Bio-Rad). Quantified data was normalized relative to the amplification of mouse GAPDH (glyceraldehyde-3-phosphate dehydrogenase) DNA.
Radiographic and gross visualization of bone lesions. A Faxitron MX20 Specimen X-ray system (Faxitron Corp., Tuscon, Ariz.) with digital exposures of 25 kV, 17 sec was used. Films were obtained on Kodak Portal Pack Oncology X-ray film (25.4×30.5 cm) for 22 kV, 15 sec. For gross pathology, bone tissues were fixed in 10% neutral buffered formalin and were decalcified for 2 h in Decal® (Decal Chemical Corp., Suffern, N.Y.) and cut in thin slices.
Statistical Analysis. For BLI error bars in graphical data represent mean±standard deviation (SD). P-values <0.05 were considered to be statistically significant.

### Results

Comparison of Cancer Specificity of AEG-Prom and PEG-Prom by Bioluminescence imaging (BLI) in PCa.

To examine the cancer-specific activity of AEG-Prom we constructed two plasmids, pAEG-Luc, expressing firefly luciferase, and pAEG-HSV1tk, expressing the herpes simplex virus type 1 thymidine kinase (not shown). AEG-Prom drives the expression of the imaging reporter genes firefly luciferase (Luc) and HSV1-tk, which enable BLI and radionuclide based-techniques, respectively. Given the high sensitivity and ease of BLI, our initial studies used this modality for proof of concept. The HSV1-tk reporter gene was used, as before (1), to provide a method that has a clear path to clinical translation. The PEG-Prom construct, namely, pPEG-Luc, was generated previously (1), and was used for the current studies.

Using BLI we tested the cancer specificity of AEG-Prom and PEG-Prom in different PCa cell lines, including PC3-ML, LNCaP, DU-145, and in the non-malignant counterpart cells of prostate epithelium. Robust expression from AEG-Prom and PEG-Prom was observed only in the malignant cell lines, whereas promoter activity was negligible in the normal prostate epithelial cells (PrEC) (FIG. 11A). The results shown in FIG. 11A are from separate experiments and are not designed to show comparative strength of the AEG-Prom and PEG-Prom, but rather to confirm selective activity in prostate cancer cells. To elucidate further the role of c-Myc in the activation of AEG-Prom, we engineered an AEG-Prom containing mutations in the two E-box elements, to which the c-Myc transcription factor was hypothesized to bind in PCa cells, to produce pAEG-mEbox1&2-Luc, similar to the one reported in Lee et al. (4) (FIG. 11B). The mutant pAEG construct, pAEG-mEbox1&2-Luc, consists of AGAGTG in lieu of the consensus CACGTG in the Ebox1 and 2 regions of the promoter. Those constructs were transiently transfected into PC3-ML cells, and the promoter activities were compared with those of the wild-type AEG-Prom construct, pAEG-Luc. As shown in FIG. 11B, the pAEG-mEbox1&2-Luc is still active in the PC3-ML cell line, although there was a seven-fold reduction in the extent of activation of the AEG-Prom construct. These results indicate that AEG-Prom activity is regulated primarily, but not exclusively, by the c-Myc transcription factor in this system.

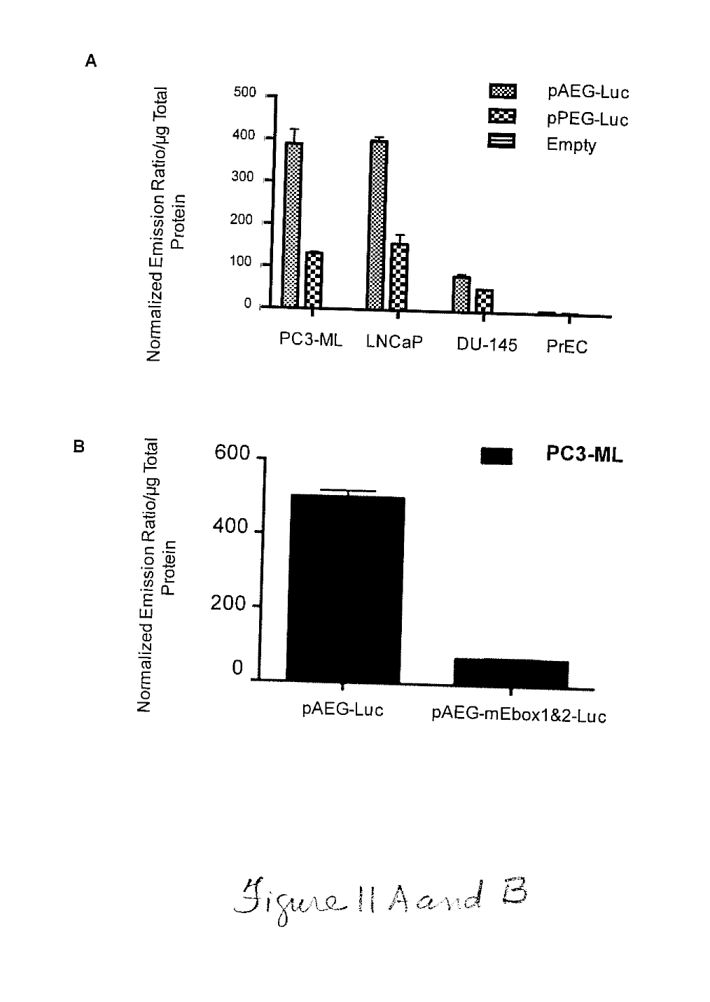

We then tested and compared the specificity of AEG-Prom and PEG-Prom in vivo in a relevant experimental model of PCa. To develop this model we used two human PCa sub-lines selected from initial metastases of the parental human PC3 cells that targeted the murine lumbar vertebrae, hence ML (metastasis lumbar). We used PC3-ML cells and the luciferase-tagged version of the PC3-ML cells, namely, PC3-ML-Luc (25-27), which were injected either intravenously (IV) or directly into the left ventricle of the heart to ensure widespread dissemination—including to bone. BLI confirmed the presence of widespread metastases after IV injection of PC3-ML-Luc cells (not shown). We assumed a similar time course for the development of metastases from the PC3-ML cells that did not express Luc so that we could use them in conjunction with the AEG-Prom-driven system to identify metastatic lesions by BLI. Mice received an IV dose of pAEG-Luc-PEI and pPEG-Luc-PEI polyplexes (FIG. 12). Twenty-four (not shown) and 48 h after plasmid DNA delivery, BLI revealed AEG-Prom- and PEG-Prom-driven gene expression above background only in the model demonstrating metastasis (FIGS. 12B and D) and not in the healthy control group (FIGS. 12A and C).

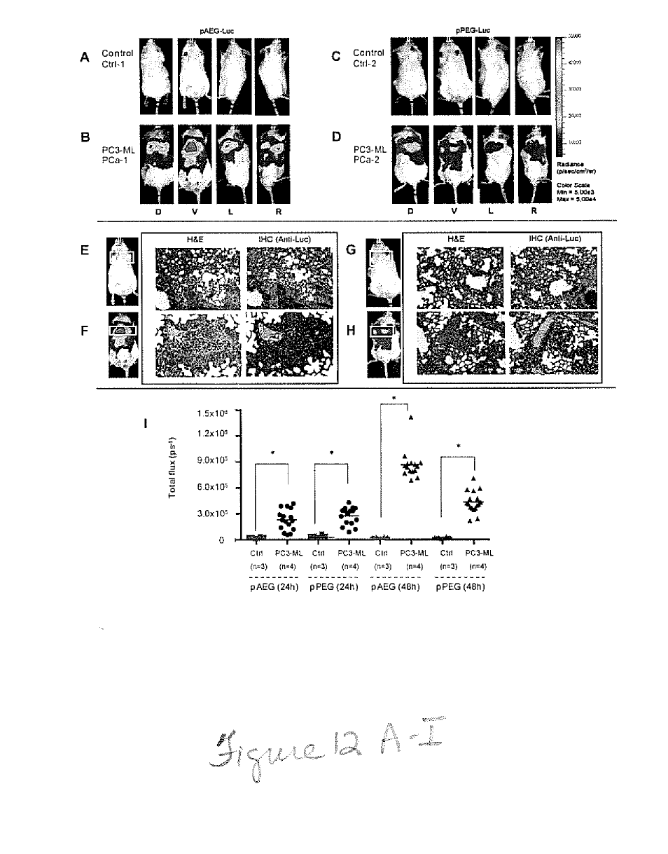

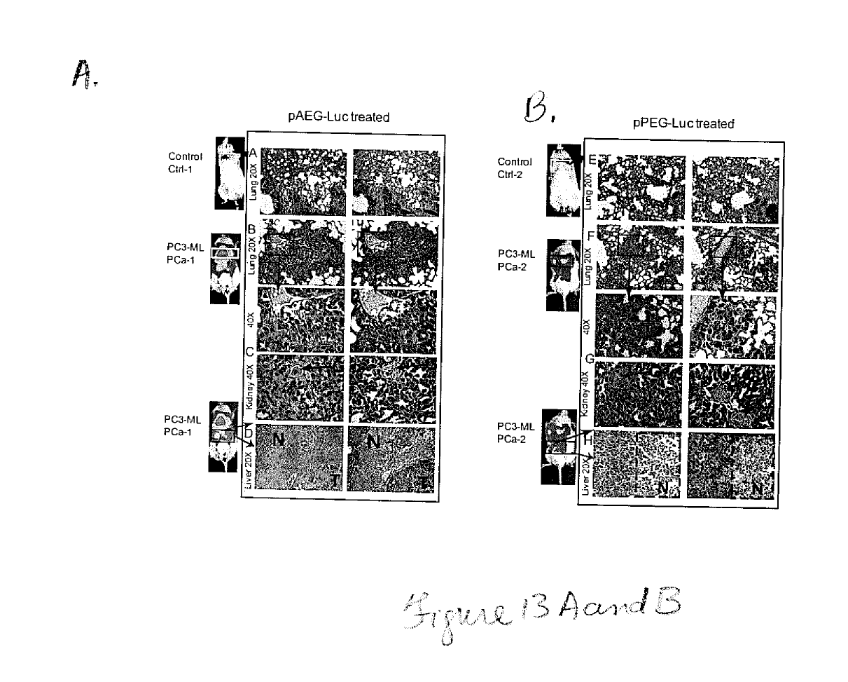

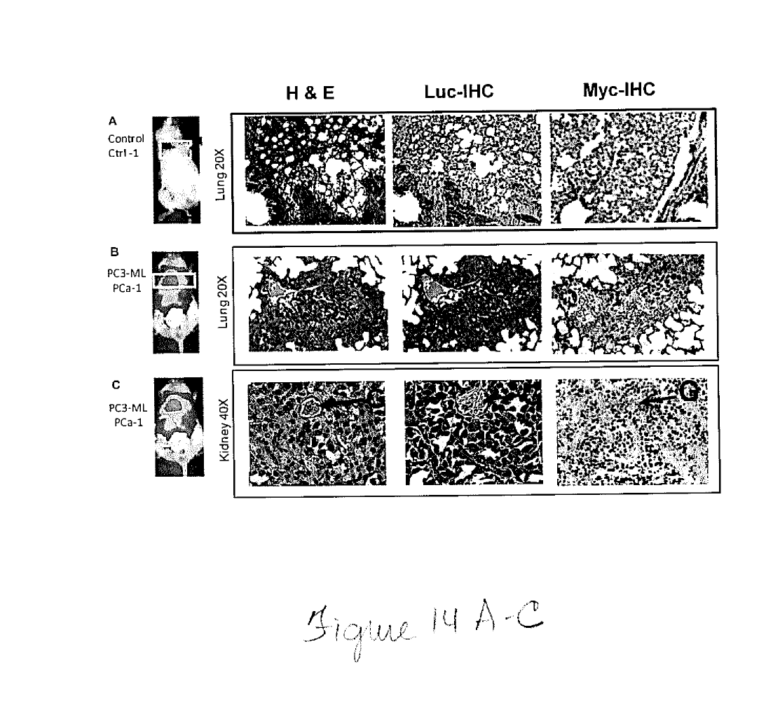

Histological analysis of the photon-emitting regions within lung for the animals treated with pAEG-Luc or pPEG-Luc showed the presence of tumor and the correlative Luc expression in the cancer models, but not in the controls (FIG. 12). In the lungs Luc expression was detected by immunohistochemistry (IHC) from uniformly scattered tumor cells with some forming large, nodular aggregates. Lesions infiltrate capillaries, interstitium, septae and larger blood vessels (FIGS. 12F and H). The kidneys also demonstrated multiple metastases. Tumor replaced all normal tissue except individual glomeruli (indicated by “G” in FIGS. 13C and G). Tumor cells in the liver formed multifocal nodules that in some cases demonstrated adjacent necrosis. Necrotic centers (indicated by “N” in FIGS. 13D and H) correlated with a lack of Luc expression. We have also shown that expression of c-myc correlates with AEG-Prom-driven Luc expression within tumor (FIG. 14). Similar expression of the c-myc or the Luc genes was not evident in the healthy, control mice.

BLI signal intensity was significantly higher in the PCa group compared to controls within lung at both the 24 and 48 h time points (after administration of pAEG-Luc and pPEG-Luc) (P<0.0001; FIG. 12I). Moreover, at the 48 h time point we observed an approximately two-fold higher Luc gene expression from the AEG-Prom group as compared to the PEG-Prom group (FIG. 12I). A possible reason for elevated expression using AEG-Prom includes that a human gene may demonstrate more productive interaction with the human proteins of the Ras-signaling pathway such as c-Myc (PEG-Prom is of rat origin) (3).

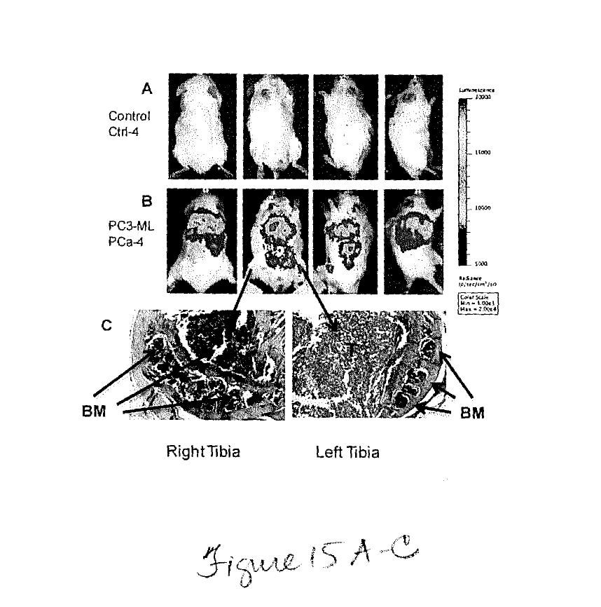

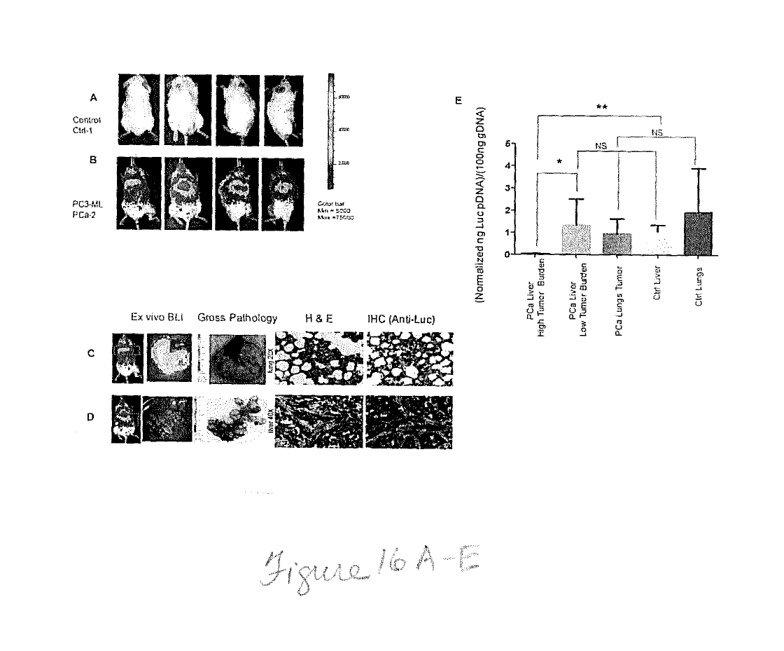

To enable reliable formation of metastasis to bone, a tissue prominently involved in human PCa, we injected PC3-ML-Luc and PC3-ML cells through an intra-cardiac route (not shown). Once timing for the development of metastases was determined for the luciferase-expressing cells, we then studied metastases due to PC3-ML cells using the pAEG-Luc-PEI polyplex. At 48 h after plasmid delivery we observed AEG-Prom-mediated expression of Luc from the PC3-ML models, as shown for PCa-4 and PCa-2 and not from controls (FIGS. 15 and 16, respectively). For PCa-4, when imaged seven weeks after cell injection, BLI was able to detect cancer cells in the left tibia (FIG. 15B), as confirmed by histological analysis (FIG. 15C). The BLI signal intensity, from deep within the bone, was weak in vivo, likely due to attenuation by living tissues (31).

Ex vivo BLI of PCa-2, when imaged five weeks after cell injection, showed the presence of tumor in the lungs, liver, adrenals and kidneys, as also confirmed by gross pathology, histological analysis and Luc IHC (FIGS. 16C and D). To study the transfection efficiencies of systemically delivered construct within lung and liver (FIG. 16E), we quantified the amount of plasmid DNA delivered to each of these tissues. We performed quantitative real-time PCR with a primer set designed to amplify a region of the Luc-encoding gene in the pAEG-Luc plasmid. We used total DNA extracted from the lung and liver as a template. The differences in transfection efficiency between areas of high tumor burden vs. those of low tumor burden within liver in same animal, as well as between areas of high tumor burden within liver vs. normal liver, were significant at P<0.0005 and P=0.0078, respectively. Lower transfection efficiency in diseased vs. normal liver was likely due to lower delivery of plasmid to the diseased tissue, as demonstrated previously for PEG-Prom and lung replete with metastases (1). PCR was also performed in tissues from control animals after pAEG-Luc delivery. Differences in transfection efficiency within lungs and liver between the control group and the PCa group were not significant. That confirms that higher visualized Luc expression from the PCa models is due to the tumor-specific activity of AEG-Prom rather than higher transfection efficiency to malignant tissues.

### Radionuclide Imaging of Cancer Via AEG-Prom.

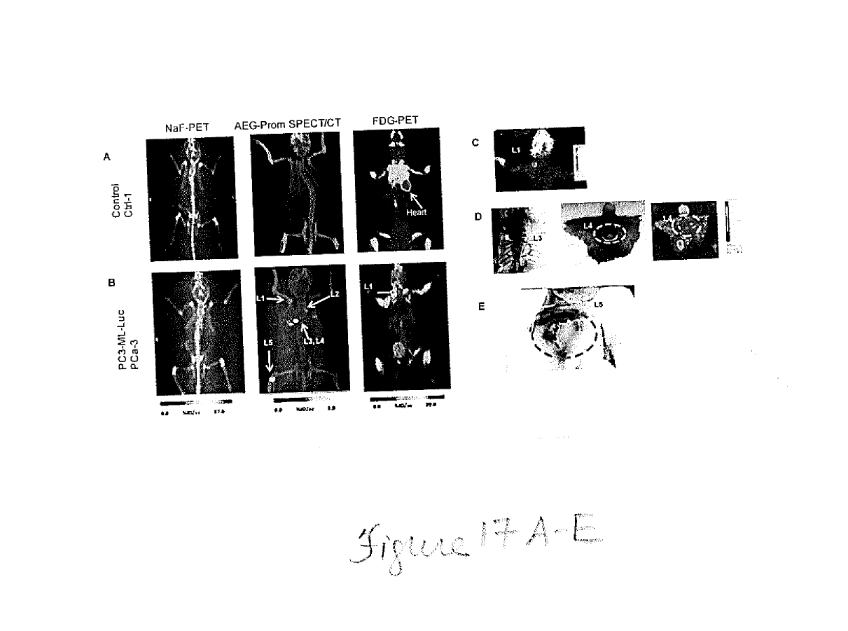

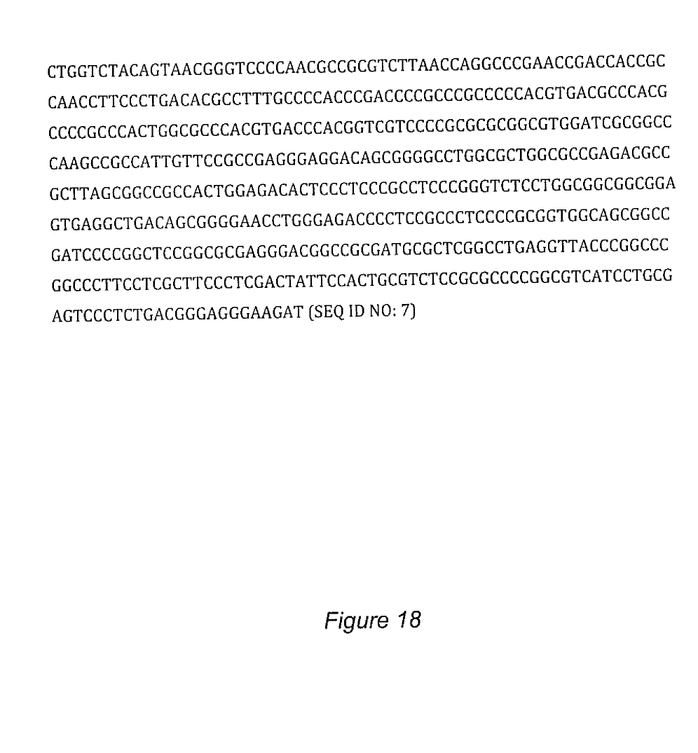

BLI is limited to pre-clinical studies due to the dependence of signal on tissue depth, the need for administration of exogenous D-luciferin substrate at relatively high concentration for light emission, rapid consumption of D-luciferin leading to unstable signal, and low anatomic resolution (1). Accordingly we cloned pAEG-HSV1tk (not shown), which can be detected by the radionuclide-based techniques of PET or single photon emission computed tomography (SPECT), upon administration of a suitably radiolabeled nucleoside analog (32). We examined the SPECT-CT imaging capabilities of pAEG-HSV1tk for detection of bone and soft tissue metastases in the PC3-ML model. Approximately five weeks after receiving an intra-cardiac administration of PC3-ML-Luc cells, the PCa group and the corresponding controls received pAEG-HSV1tk-PEI polyplex. Forty-eight hours after plasmid delivery, mice received the known HSV1-TK substrate, 2′-fluoro-2′-deoxy-β-d-5-[¹²⁵I]iodouracil-arabinofuranoside ([¹²⁵I]FIAU) (29), and were imaged at 18-20 h after injection of radiotracer. FIG. 17 shows a representative example, PCa-3, for which we were able to detect the presence of multiple metastatic lesions with the pAEG-HSV1tk system. We then compared the sensitivity of the AEG-Prom imaging system to the current clinical PET-based methods for detecting soft tissue (FDG-PET) and bony (NaF-PET) metastatic lesions due to PCa. FIGS. 17 A and B shows a representative example, PCa-3, and a healthy control, Ctrl-1, imaged with each method. Because NaF is a bone-seeking agent, there is substantial uptake within the normal skeleton (33), which may obscure lesions within bone (FIG. 17A). Moreover, on NaF bone scan skeletal metastases are seen indirectly, depending on the reaction of bone to the lesion, while the AEG-Prom polyplex images tumor directly. The NaF-PET/CT study for PCa-3 appears similar to that for Ctrl-1. NaF-PET/CT failed to identify the metastases within tibia and axial skeleton. The same mouse also underwent FDG-PET/CT, which was only able to identify a lesion in the left shoulder (L1, FIG. 17B).

BLI performed ex vivo and gross pathology of lesions within the right shoulder, dorsal thoracic wall, ribs, sternum and the heart confirmed that tumor was the source of signal seen on the living images (FIGS. 17C and E). We were able to identify a 3 mm tumor nodule on the heart (L2), a 5 mm lesion in the dorsal thoracic wall adjacent to the mid-spine (L3, FIG. 17D) and a 1 mm lesion in the ventral midline of the sternum (L4, FIG. 17D). Furthermore, the macroscopic picture confirmed metastases in the bone marrow within proximal tibia (L5, FIG. 17E, dotted circles). An ex vivo plain film image of the pelvis of PCa-3 did not delineate bone lesions clearly (not shown), suggesting advanced changes in skeletal morphology might be needed for detection with conventional imaging modalities.

### Discussion

Our goal was to develop a systemically deliverable construct for molecular-genetic imaging of metastatic lesions within both soft tissue and bone in a relevant model of PCa. Ultimately the intent of this technology will be to utilize a radionuclide-based imaging system for clinical translation. As stated at the outset, current clinical methods to image PCa are sub-optimal, and a genetic method, which can be paired with concurrent therapy (theranostic), would be a useful addition to detecting (and treating) the disease.

The tumor-specific promoters of PEG-3 and AEG-1 have certain features that might render them more specific and selective while at the same time instill them with greater utility than other promoters, namely to use them in a variety of cancers beyond PCa. PEG-Prom and AEG-Prom: [1] maintain universal cancer specificity regardless of the tissue of origin; [2] do not require amplification to achieve high sensitivity; and, [3] are systemically delivered using a non-viral delivery vehicle. To recapitulate the clinical characteristics of PCa metastasis, we implemented a bone metastatic model of PCa in which we tested AEG-Prom activity. In animals showing tibial lesions using BLI of PC3-ML-Luc cells, subsequent SPECT/CT was able to detect these lesions in all animals tested (FIG. 17).

By using a biodegradable polymer, in vivo-jetPEI®, we addressed certain problems that may arise when employing viral vectors, such as immune-mediated toxicity, inflammation and liver tropism. We checked the ability of the non-viral delivery vehicle to provide widespread, systemic dissemination of plasmid by conducting quantitative PCR on sections of lung and liver and compared the transfection efficiency between controls and animals affected with PCa (FIG. 16). Group differences in transfection efficiency between lung and liver were insignificant. This study also confirmed our earlier results that nanoparticle delivery is most efficient to well-vascularized tissues (1). Liver tissue sections with a high tumor burden had significantly lower (P<0.005) delivery of plasmid DNA, possibly due to the reduced vasculature of this tissue, which was also likely under high hydrostatic pressure and was adjacent to necrotic areas. Although imaging was mediated through activation of AEG-Prom, delivery was in part mediated through the enhanced permeability and retention (EPR) effect and endocystosis (35).

AEG-Prom enables a sensitive method for molecular-genetic imaging of PCa in vivo. From mutational analysis of AEG-Prom we have shown that its activation relies significantly on c-Myc binding to the two E-box elements discussed above. As Ras-mediated c-Myc signal transduction is a pathway present in nearly all malignancies yet is absent in normal tissue (36), AEG-Prom will enable imaging of a wide variety of cancers directly and specifically.

### REFERENCES for Example 3

1. Bhang H-eC, Gabrielson K L, Laterra J, Fisher P B, Pomper M G. Tumor-specific imaging through progression elevated gene-3 promoter-driven gene expression. Nature medicine. 2011; 17:123-U302.
2. Kubo H, Gardner T A, Wada Y, Koeneman K S, Gotoh A, Yang L, et al. Phase I dose escalation clinical trial of adenovirus vector carrying osteocalcin promoter-driven herpes simplex virus thymidine kinase in localized and metastatic hormone-refractory prostate cancer. Human gene therapy. 2003; 14:227-41.
3. Su Z Z, Sarkar D, Emdad L, Duigou G J, Young C S, Ware J, et al. Targeting gene expression selectively in cancer cells by using the progression-elevated gene-3 promoter. Proceedings of the National Academy of Sciences of the United States of America. 2005; 102:1059-64.
4. Lee S G, Su Z Z, Emdad L, Sarkar D, Fisher P B. Astrocyte elevated gene-1 (AEG-1) is a target gene of oncogenic Ha-ras requiring phosphatidylinositol 3-kinase and c-Myc. Proceedings of the National Academy of Sciences of the United States of America. 2006; 103:17390-5.
5. Su Z Z, Kang D C, Chen Y, Pekarskaya O, Chao W, Volsky D J, et al. Identification and cloning of human astrocyte genes displaying elevated expression after infection with HIV-1 or exposure to HIV-1 envelope glycoprotein by rapid subtraction hybridization, RaSH. Oncogene. 2002; 21:3592-602.
6. Lee S G, Kang D C, DeSalle R, Sarkar D, Fisher P B. AEG-1/MTDH/LYRIC, the beginning: initial cloning, structure, expression profile, and regulation of expression. Advances in cancer research. 2013; 120:1-38.
7. Brown D M, Ruoslahti E. Metadherin, a cell surface protein in breast tumors that mediates lung metastasis. Cancer cell. 2004; 5:365-74.
8. Britt D E, Yang D F, Yang D Q, Flanagan D, Callanan H, Lim Y P, et al. Identification of a novel protein, LYRIC, localized to tight junctions of polarized epithelial cells. Exp Cell Res. 2004; 300:134-48.
9. Emdad L, Lee S G, Su Z Z, Jeon H Y, Boukerche H, Sarkar D, et al. Astrocyte elevated gene-1 (AEG-1) functions as an oncogene and regulates angiogenesis. Proceedings of the National Academy of Sciences of the United States of America. 2009; 106:21300-5.
10. Sarkar D, Fisher P B. AEG-1/MTDH/LYRIC: clinical significance. Advances in cancer research. 2013; 120:39-74.
11. Emdad L, Das S K, Dasgupta S, Hu B, Sarkar D, Fisher P B. AEG-1/MTDH/LYRIC: signaling pathways, downstream genes, interacting proteins, and regulation of tumor angiogenesis. Advances in cancer research. 2013; 120:75-111.
12. Thirkettle H J, Mills I G, Whitaker H C, Neal D E. Nuclear LYRIC/AEG-1 interacts with PLZF and relieves PLZF-mediated repression. Oncogene. 2009; 28:3663-70.
13. Liu I J, Zafar M B, Lai Y H, Segall G M, Terris M K. Fluorodeoxyglucose positron emission tomography studies in diagnosis and staging of clinically organ-confined prostate cancer. Urology. 2001; 57:108-11.
14. Grant F D, Fahey F H, Packard A B, Davis R T, Alavi A, Treves S T. Skeletal PET with 18F-Fluoride: Applying New Technology to an Old Tracer. J Nucl Med. 2008; 49:68-78.
15. Yen R F, Chen C Y, Cheng M F, Wu Y W, Shiau Y C, Wu K, et al. The diagnostic and prognostic effectiveness of F-18 sodium fluoride PET-CT in detecting bone metastases for hepatocellular carcinoma patients. Nuclear medicine communications. 2011; 31:637-45.
16. Kwee S A, Wei H, Sesterhenn I, Yun D, Coel M N. Localization of primary prostate cancer with dual-phase 18F-fluorocholine PET. J Nucl Med. 2006; 47:262-9.
17. Kwee S A, Thibault G P, Stack R S, Coel M N, Furusato B, Sesterhenn I A. Use of step-section histopathology to evaluate 18F-fluorocholine PET sextant localization of prostate cancer. Mol Imaging. 2008; 7:12-20.
18. Piert M, Park H, Khan A, Siddiqui J, Hussain H, Chenevert T, et al. Detection of aggressive primary prostate cancer with 11C-choline PET/CT using multimodality fusion techniques. J Nucl Med. 2009; 50:1585-93.
19. Beattie B J, Smith-Jones P M, Jhanwar Y S, Schoder H, Schmidtlein C R, Morris M J, et al. Pharmacokinetic assessment of the uptake of 16beta-18F-fluoro-5alpha-dihydrotestosterone (FDHT) in prostate tumors as measured by PET. J Nucl Med. 2010; 51:183-92.
20. Schuster D M, Savir-Baruch B, Nieh P T, Master V A, Halkar R K, Rossi P J, et al. Detection of recurrent prostate carcinoma with anti-1-amino-3-18F-fluorocyclobutane-1-carboxylic acid PET/CT and 111In-capromab pendetide SPECT/CT. Radiology. 2011; 259:852-61.
21. Cho S Y, Gage K L, Mease R C, Senthamizhchelvan S, Holt D P, Jeffrey-Kwanisai A, et al. Biodistribution, tumor detection, and radiation dosimetry of 18F-DCFBC, a low-molecular-weight inhibitor of prostate-specific membrane antigen, in patients with metastatic prostate cancer. J Nucl Med. 2012; 53:1883-91.
22. Sidi A A, Ohana P, Benjamin S, Shalev M, Ransom J H, Lamm D, et al. Phase I/II marker lesion study of intravesical BC-819 DNA plasmid in H19 over expressing superficial bladder cancer refractory to bacillus Calmette-Guerin. J Urol. 2008; 180:2379-83.
23. Lisziewicz J, Trocio J, Whitman L, Varga G, Xu J, Bakare N, et al. DermaVir: a novel topical vaccine for HIV/AIDS. The Journal of investigative dermatology. 2005; 124:160-9.
24. Lisziewicz J, Bakare N, Calarota S A, Banhegyi D, Szlavik J, Ujhelyi E, et al. Single DermaVir immunization: dose-dependent expansion of precursor/memory T cells against all HIV antigens in HIV-1 infected individuals. PloS one. 2012; 7:e35416.
25. Wang M, Steams M E. Isolation and characterization of PC-3 human prostatic tumor sublines which preferentially metastasize to select organs in S.C.I.D. mice. Differentiation. 1991; 48:115-25.
26. Russell M R, Jamieson W L, Dolloff N G, Fatatis A. The alpha-receptor for platelet-derived growth factor as a target for antibody-mediated inhibition of skeletal metastases from prostate cancer cells. Oncogene. 2009; 28:412-21.
27. Lynch T P, Ferrer C M, Jackson S R, Shahriari K S, Vosseller K, Reginato M J. Critical role of O-Linked beta-N-acetylglucosamine transferase in prostate cancer invasion, angiogenesis, and metastasis. The Journal of biological chemistry. 2012; 287:11070-81.
28. Adams J Y, Johnson M, Sato M, Berger F, Gambhir S S, Carey M, et al. Visualization of advanced human prostate cancer lesions in living mice by a targeted gene transfer vector and optical imaging. Nature medicine. 2002; 8:891-7.
29. Hong H, Yang Y, Cai W. Imaging gene expression in live cells and tissues. Cold Spring Harbor protocols. 2011; 2011:pdb top103.
30. Loening A M, Gambhir S S. AMIDE: a free software tool for multimodality medical image analysis. Mol Imaging. 2003; 2:131-7.
31. Wu J C, Sundaresan G, Iyer M, Gambhir S S. Noninvasive optical imaging of firefly luciferase reporter gene expression in skeletal muscles of living mice. Mol Ther. 2001; 4:297-306.
32. Jacobs A, Voges J, Reszka R, Lercher M, Gossmann A, Kracht L, et al. Positron-emission tomography of vector-mediated gene expression in gene therapy for gliomas. Lancet. 2001; 358:727-9.
33. Czernin J, Satyamurthy N, Schiepers C. Molecular mechanisms of bone 18F-NaF deposition. J Nucl Med. 2010; 51:1826-9.
34. Wu L, Matherly J, Smallwood A, Adams J Y, Billick E, Belldegrun A, et al. Chimeric PSA enhancers exhibit augmented activity in prostate cancer gene therapy vectors. Gene therapy. 2001; 8:1416-26.
35. Kirtane A R, Panyam J. Polymer nanoparticles: Weighing up gene delivery. Nature nanotechnology. 2013; 8:805-6.
36. Ischenko I, Zhi J, Moll U M, Nemajerova A, Petrenko O. Direct reprogramming by oncogenic Ras and Myc. Proceedings of the National Academy of Sciences of the United States of America. 2013; 110:3937-42.

All terms and phrases (e.g. nucleic acid, protein, polypeptide, etc.) used herein have the meaning as commonly understood in the art, unless otherwise indicated.

As used in this specification and the appended claims, the singular forms “a,” “an” and “the” include plural references unless the content clearly dictates otherwise.

All patents, patent applications and publications cited herein, whether supra or infra, are hereby incorporated by reference in their entirety.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of imaging tumors or cancerous cells or tissue that arise from spontaneous metastasis in a subject, comprising the steps of
administering to said subject a nucleic acid construct comprising
an imaging reporter gene operably linked to a cancer specific or cancer selective promoter;
administering to said subject an imaging agent that is complementary to said imaging reporter gene; and
imaging tumors or cancerous tissues or cells that arise from spontaneous metastasis in said subject by detecting a detectable signal from said imaging agent.

2. The method of claim 1, wherein said imaging reporter gene is selected from the group consisting of luciferase, herpes simplex virus 1 thymidine kinase (HSV1-tk), and sodium-iodide symporter.

3. The method of claim 1, wherein said imaging reporter gene is HSV1-tk and said subject is a cancer patient.

4. The method of claim 1, wherein said imaging agent is a radiolabeled nucleoside analog.

5. The method of claim 4, wherein said radiolabeled nucleoside analog is 2′-fluoro-2′deoxy-β-D-5-[¹²⁵I]iodouraci 1-arabinofuranoside,

6. The method of claim 1, wherein said step of imaging is carried out via single photon emission computed tomography (SPECT) or by positron emission tomography (PET)

7. The method of claim 1, wherein said nucleic acid construct is present in a polyplex with a cationic polymer.

8. The method of claim 7, wherein said cationic polymer is polyethylemeinine.

9. The method of claim 1, wherein said step of administering a nucleic acid construct is carried out by intravenous injection.

10. The method of claim 1, wherein said tumors, cancerous tissues or cells that arise from spontaneous metastasis include cancer cells of a type selected from groups consisting of breast cancer, melanoma, carcinoma of unknown primary (CUP), neuroblastoma, malignant glioma, cervical, colon, hepatocarcinoma, ovarian, lung, pancreatic, and prostate cancer.

11. The method of claim 1, wherein said nucleic acid construct is present in a plasmid.

12. The method of claim 1, wherein said nucleic acid construct is present in a viral vector.

13. The method of claim 14, wherein said viral vector is a conditionally replication-competent adenovirus.

14. The method of claim 1, wherein said cancer specific or cancer selective promoter is selected from the group consisting of progression elevated gene-3 (PEG-3) promoter and astrocyte elevated gene 1 (AEG-1) promoter.

15. The method of claim 1, wherein at least one step of said administering steps is carried out systemically.

16. A method of both imaging and treating tumors, or cancerous tissues or cells that arise from spontaneous metastasis in a subject, comprising the steps of
administering to said subject one or more nucleic acid constructs comprising
an imaging reporter gene operably linked to a cancer specific or cancer selective promoter and a gene encoding an anti-tumor agent;
administering to said subject an imaging agent that is complementary to said imaging reporter gene; and
imaging tumors or cancerous tissues or cells that arise from spontaneous metastasis in said subject by detecting a detectable signal from said imaging agent, wherein said gene encoding said anti-tumor agent is expressed by cells in said tumors or cancerous tissues or cells to act on said cells.

17. The method of claim 16, wherein said gene encoding an anti-tumor agent is operably linked to a tandem gene expression element.

18. The method of claim 17, wherein said tandem gene expression element is an internal ribosomal entry site (IRES).

19. The method of claim 16, wherein said gene encoding an anti-tumor agent is operably linked to a cancer specific or cancer selective promoter.

20. The method of claim 16, wherein said anti-tumor agent is mda-7/IL-24 or tumor necrosis factor (TNF)-related apoptosis-inducing ligand (TRAIL).

21. The method of claim 16, wherein at least one of said administering steps is carried out systemically.

22. A method of treating or preventing spontaneous metastasis in a patient in need thereof, comprising
administering to said subject one or more nucleic acid constructs comprising
a gene encoding an anti-tumor agent operably linked to a cancer specific or cancer selective promoter, wherein said gene encoding said anti-tumor agent is specifically or selectively expressed by cancer cells in amounts sufficient to damage or kill said cancer cells.

23. The method of claim 22, wherein said construct further comprises
an imaging reporter gene operably linked to a cancer specific or cancer selective promoter.

24. The method of claim 23, further comprising a steps of
administering to said subject an imaging agent that is complementary to said imaging reporter gene; and
imaging tumors or cancerous tissues or cells in said subject by detecting a detectable signal from said imaging agent.
